(19) United States
(12) Patent Application Publication
(10) Pub. No.: US 2026/0028343 A1
(43) Pub. Date: Jan. 29, 2026

(54) Substituted Imidazopyrazine Compounds as Ligand Directed Degraders of IRAK3

(71) Applicant: Celgene Corporation, Princeton, NJ (US)

(72) Inventors: Farid van der Mei, Cambridge, MA (US); Guobin Miao, Lexington, MA (US); Rulin Ma, Winchester, MA (US); Laura Akullian D'Agostino, Sudbury, MA (US); Kurt Armbrust, Cambridge, MA (US)

(73) Assignee: Celgene Corporation, Princeton, NJ (US)

(21) Appl. No.: 18/997,383

(22) PCT Filed: Jul. 24, 2023
(86) PCT No.: PCT/US23/70819

§ 371 Date: Jan. 21, 2025

Related U.S. Application Data
(60) Provisional application No. 63/391,969, filed on Jul. 25, 2022.

(51) Int. Cl. C07D 487/04; A61K 31/4985

(52) U.S. Cl. CPC C07D 487/04; A61K 31/4985

(57) ABSTRACT

Provided herein are compounds and compositions thereof for modulating IRAK3. In some embodiments, the compounds and compositions are provided for treatment of cancer.

### CROSS-REFERENCE TO RELATED APPLICATIONS

This application claims priority to U.S. Provisional Application No. 63/391,969, filed on Jul. 25, 2022, the disclosure of which is incorporated herein by reference in its entirety for any purpose.

### FIELD

The present disclosure relates generally to compounds, compositions, and methods for their preparation and use of the compounds and compositions, e.g., for treating cancer.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian IRAK family, TRAK2 and IRAK3 are thought to be catalytically inactive pseudokinases (Wesche et al., J. Biol. Chem. 1999, 274, 19403-19410), but the detailed roles of the two kinases are still largely unknown (Lagne et al., Structure 2021, 29, 238-251). Nonetheless, reports indicate the association of IRAK3 with negative regulation of TLR (toll-like receptor) signaling which is involved in detecting microorganisms and protecting multicellular organisms from infection (Kobayashi et al., Cell 2002, 110, 191-202). More recent studies have revealed the linkage between mutation or high expression levels of IRAK3 and various diseases such as asthma and cancer (Balaci et al., Am. J. Hum. Genet. 2007, 80 (6), 1103-1114; Kesselring, R. Cancer Cell 2016, 29 (5), 685-696), which suggests the potential of IRAK3 as a drug target and the need for IRAK3 binding small molecules.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK3 for degradation.

### SUMMARY

Described herein, in certain embodiments, are compounds and compositions thereof for degrading IRAK3. In various embodiments, the compounds and compositions thereof may be used for treatment of cancer. In various embodiments, the compounds and compositions thereof may be used for enhancing immunity in a subject receiving a vaccine.

The present embodiments can be understood more fully by reference to the detailed description and examples, which are intended to exemplify non-limiting embodiments.

Embodiment A1. A compound of Formula (I):

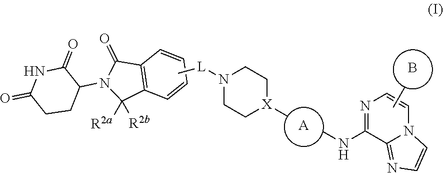

or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
Ring B is C₆-C₁₀ aryl or C₃-C₆ cycloalkyl, each of which is optionally substituted by 1-5 substituents selected from C₁-C₆ alkyl, C₁-C₆ haloalkyl, C₁-C₆ alkoxy, halo, —OH, and —CN;
X is CH or N;
L is —O(C₁-C₆ alkylene)C(O)—, —O(C₁-C₆ alkylene)C(O)NR¹(C₁-C₆ alkylene)C(O)—, —O(C₁-C₆ alkylene)C(O)NR¹(C₁-C₆ alkylene)-O—(C₁-C₆ alkylene)C(O)—, —O(C₁-C₆ alkylene)C(O)NR¹(C₁-C₆ alkylene)-, or —C(O)(6- to 11-membered spiro heterocyclene)-;
R¹ is H or C₁-C₆ alkyl; and
R^{2a} and R^{2b} are each H or are taken together to form an oxo group.

Embodiment A2. The compound of embodiment A1, or a pharmaceutically acceptable salt thereof, wherein:
Ring A is C₆-C₁₀ aryl.

Embodiment A3. The compound of embodiment A1, or a pharmaceutically acceptable salt thereof, wherein:

Ring A is 5- to 6-membered heteroaryl, wherein the heteroaryl contains 1-3 nitrogens.

Embodiment A4. The compound of embodiment A1 or A2, or a pharmaceutically acceptable salt thereof, wherein:

### Ring A is

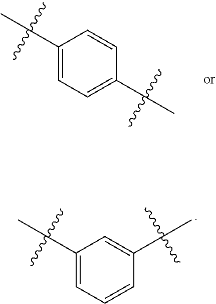 [SMILES: *c1ccc(*)cc1 ; *c1cccc(*)c1]

Embodiment A5. The compound of embodiment A1 or A3, or a pharmaceutically acceptable salt thereof, wherein:

### Ring A is

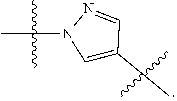 [SMILES: *c1cnn(*)c1]

Embodiment A6. The compound of any one of embodiments A1-A5, or a pharmaceutically acceptable salt thereof, wherein Ring B is C₆-C₁₀ aryl optionally substituted by 1-5 substituents selected from C₁-C₆ alkyl.

Embodiment A7. The compound of embodiment A6, or a pharmaceutically acceptable salt thereof, wherein Ring B is C₆-C₁₀ aryl optionally substituted by 1-3 substituents selected from C₁-C₃ alkyl.

Embodiment A8. The compound of any one of embodiments A1-A7, or a pharmaceutically acceptable salt thereof, wherein:

### Ring B is

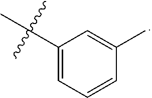 [SMILES: *c1cccc(C)c1]

Embodiment A9. The compound of any one of embodiments A1-A8, or a pharmaceutically acceptable salt thereof, wherein X is CH.

Embodiment A10. The compound of any one of embodiments A1-A8, or a pharmaceutically acceptable salt thereof, wherein X is N.

Embodiment A1 l. The compound of any one of embodiments A1-A10, or a pharmaceutically acceptable salt thereof, wherein:
L is —O(C₁-C₃ alkylene)C(O)—, —O(C₁-C₃ alkylene)C(O)NR¹(C₁-C₃ alkylene)C(O)—, —O(C₁-C₃ alkylene)C(O)NR¹(C₁-C₃ alkylene)-O—(C₁-C₃ alkylene)C(O)—, —O(C₁-C₃ alkylene)C(O)NR¹(C₁-C₃ alkylene)-, or —C(O)(10- to 11-membered spiro heterocyclene)-; and
R¹ is H.

Embodiment A12. The compound of any one of embodiments A1-A11, or a pharmaceutically acceptable salt thereof, wherein L is:

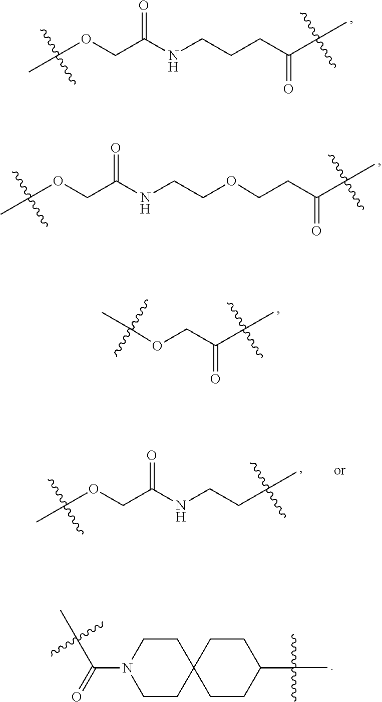 [SMILES: *OCC(=O)NCCCC(*)=O ; *OCC(=O)NCCOCCC(*)=O ; *OCC(*)=O ; *CCNC(=O)CO* ; *C(=O)N1CCC2(CCC(*)CC2)CC1]

Embodiment A13. The compound of any one of embodiments A1-A12, or a pharmaceutically acceptable salt thereof, wherein R^{2a} and R^{2b} are each H.

Embodiment A14. The compound of any one of embodiments A1-A12, or a pharmaceutically acceptable salt thereof, wherein R^{2a} and R^{2b} are taken together to form an oxo group.

Embodiment A15. The compound of any one of embodiments A1-A14, or a pharmaceutically acceptable salt thereof, wherein the compound is Formula (II), (IIIa), (IIIb), or (IV):

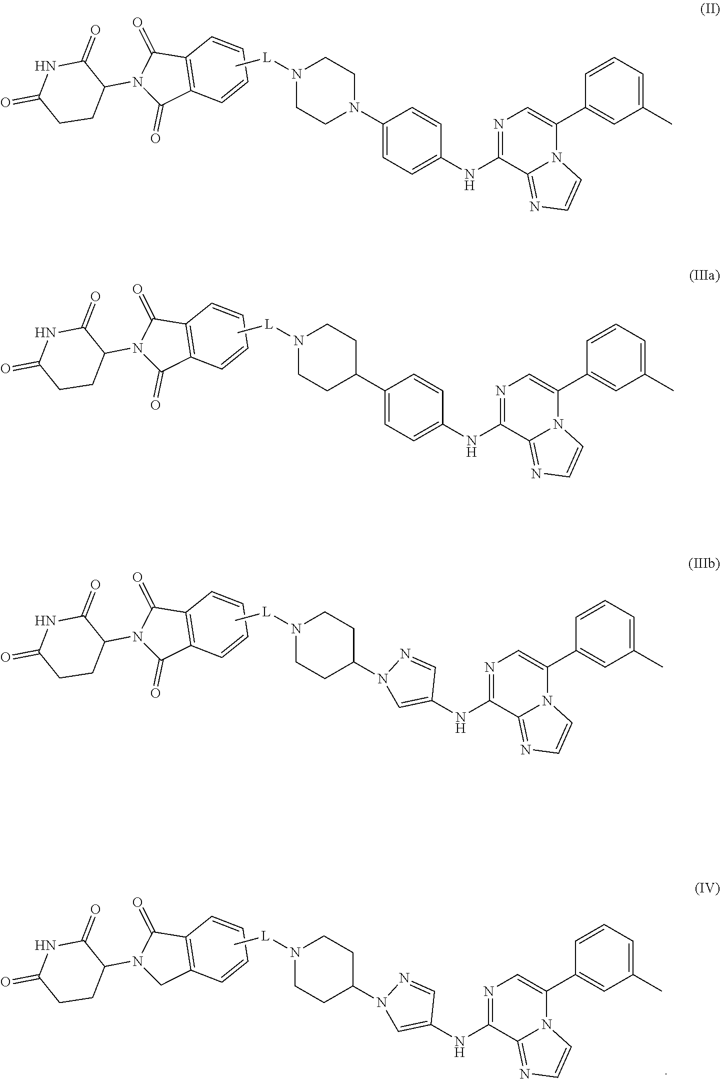

Embodiment A16. A compound selected from the compounds of Table 1 or a pharmaceutically acceptable salt thereof.

Embodiment A17. A pharmaceutical composition comprising the compound of any one of embodiments A1-A16, or a pharmaceutically acceptable salt thereof, and a pharmaceutically acceptable excipient.

Embodiment A18. A method of modulating Interleukin-1 Receptor-Associated Kinase 3 (IRAK3) comprising contacting IRAK3 with an effective amount of the compound of any one of embodiments A1-A16, or a pharmaceutically acceptable salt thereof, or the pharmaceutical composition of embodiment A17.

Embodiment A19. A method of treating cancer in a subject in need thereof, comprising administering to the subject an effective amount of the compound of any one of embodiments A1-A16, or a pharmaceutically acceptable salt thereof, or the pharmaceutical composition of embodiment A17, optionally wherein the cancer is selected from bladder cancer, breast cancer, esophageal cancer, colon cancer, head and neck cancer, kidney cancer, lung cancer, pancreatic cancer, prostate cancer, melanoma, and gastric cancer.

Embodiment A20. A method of enhancing immunity in a subject receiving a vaccine, comprising administering to the subject an effective amount of the compound of any one of embodiments A1-A16, or a pharmaceutically acceptable salt thereof, or the pharmaceutical composition of embodiment A17.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkylene” group refers to the same residues as alkyl, but having bivalency. Particular alkylene groups are those having from 1 to 10 carbon atoms (C₁-C₁₀ alkylene), typically from 1 to 8 carbons (C₁-C₈ alkylene) or, in some embodiments, from 1 to 6 (C₁-C₆ alkylene) or 1 to 3 (C₁-C₃ alkylene) carbon atoms. Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₅ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heterocyclylene” group refers to a divalent “heterocyclyl” group.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “halogen” or “halo” is fluorine, chlorine, bromine or iodine.

An “alkoxy” group is —O-(alkyl), wherein alkyl is defined above.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of Formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

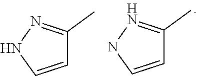

As readily understood by one skilled in the art, a wide variety of functional groups and other stuctures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (2H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an IRAK3 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

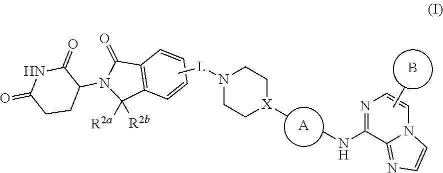

or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
Ring B is C₆-C₁₀ aryl or C₃-C₆ cycloalkyl, each of which is optionally substituted by 1-5 substituents selected from C₁-C₆ alkyl, C₁-C₆ haloalkyl, C₁-C₆ alkoxy, halo, —OH, and —CN;
X is CH or N;
L is —O(C₁-C₆ alkylene)C(O)—, —O(C₁-C₆ alkylene)C(O)NR¹(C₁-C₆ alkylene)C(O)—, —O(C₁-C₆ alkylene)C(O)NR¹(C₁-C₆ alkylene)-O—(C₁-C₆ alkylene)C(O)—, —O(C₁-C₆ alkylene)C(O)NR¹(C₁-C₆ alkylene)-, or —C(O)(6- to 11-membered spiro heterocyclene)-;
R¹ is H or C₁-C₆ alkyl; and
R^{2a} and R^{2b} are each H or are taken together to form an oxo group.

In some embodiments, Ring A is C₆-C₁₀ aryl or 5- to 6-membered heteroaryl, wherein the heteroaryl contains 1-3 heteroatoms selected from nitrogen, oxygen, and sulfur. In some embodiments, Ring A is C₆-C₁₀ aryl or 5- to 6-membered heteroaryl, wherein the heteroaryl contains 2-3 heteroatoms selected from nitrogen, oxygen, and sulfur. In some embodiments, Ring A is phenyl or 5-membered heteroaryl, wherein the heteroaryl contains 2-3 heteroatoms selected from nitrogen, oxygen, and sulfur. In some embodiments, Ring A is phenyl or 5-membered heteroaryl, wherein the heteroaryl contains 2-3 nitrogen atoms. In some embodiments, Ring A is phenyl. In some embodiments, Ring A is 5-membered heteroaryl containing 2-3 nitrogen atoms.

In some embodiments, Ring A is C₆-C₁₀ aryl. In some embodiments, Ring A is C₆ aryl. In some embodiments, Ring A is phenyl. In some embodiments, Ring A is C₁₀ aryl. In some embodiments, Ring A is C₆-C₁₀ naphthyl. In some embodiments, Ring A is

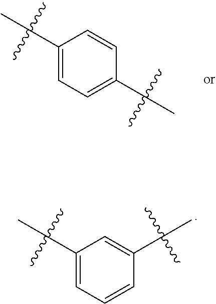 [SMILES: *c1ccc(*)cc1 ; *c1cccc(*)c1]

In some embodiments, Ring A is 5- to 6-membered heteroaryl containing 1-3 heteroatoms selected from nitrogen, oxygen, and sulfur. In some embodiments, Ring A is 5- to 6-membered heteroaryl containing one heteroatom selected from nitrogen, oxygen, and sulfur. In some embodiments, Ring A is 5- to 6-membered heteroaryl containing 2-3 heteroatoms selected from nitrogen, oxygen, and sulfur. In some embodiments, Ring A is 5- to 6-membered heteroaryl containing 2 heteroatoms selected from nitrogen, oxygen, and sulfur. In some embodiments, Ring A is 5- to 6-membered heteroaryl containing 3 heteroatoms selected from nitrogen, oxygen, and sulfur. In some embodiments, Ring A is 5-membered heteroaryl containing one heteroatom selected from nitrogen, oxygen, and sulfur. In some embodiments, Ring A is 5-membered heteroaryl containing 2-3 heteroatoms selected from nitrogen, oxygen, and sulfur. In some embodiments, Ring A is 5-membered heteroaryl containing 2 nitrogen atoms. In some embodiments, Ring A is 5-membered heteroaryl containing 3 nitrogen atoms. In some embodiments, Ring A is 5-membered heteroaryl containing one nitrogen atom and one sulfur atom. In some embodiments, Ring A is 5-membered heteroaryl containing one nitrogen atom and one oxygen atom. In some embodiments, Ring A is pyrrolyl, pyrazolyl, triazolyl, imidazolyl, thiazolyl, oxazolyl, isoxazolyl, furanyl, or thiadiazolyl. In some embodiments, Ring A is 6-membered heteroaryl containing one heteroatom selected from nitrogen, oxygen, and sulfur. In some embodiments, Ring A is 6-membered heteroaryl containing 2-3 heteroatoms selected from nitrogen, oxygen, and sulfur. In some embodiments, Ring A is 6-membered heteroaryl containing 2 heteroatoms selected from nitrogen, oxygen, and sulfur. In some embodiments, Ring A is 6-membered heteroaryl containing 3 heteroatoms selected from nitrogen, oxygen, and sulfur. In some embodiments, Ring A is 6-membered heteroaryl containing 2 nitrogen atoms. In some embodiments, Ring A is 6-membered heteroaryl containing 3 nitrogen atoms. In some embodiments, Ring A is 6-membered heteroaryl containing one nitrogen atom and one sulfur atom. In some embodiments, Ring A is 6-membered heteroaryl containing one nitrogen atom and one oxygen atom. In some embodiments, Ring A is pyridinyl, pyrazinyl, pyrimidinyl, pyridazinyl, or triazinyl. In some embodiments, Ring A is

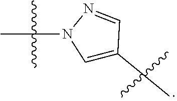 [SMILES: *c1cnn(*)c1]

In some embodiments, Ring B is C₆-C₁₀ aryl or C₃-C₆ cycloalkyl, each of which is optionally substituted by 1-5 substituents selected from C₁-C₆ alkyl, C₁-C₆ haloalkyl, C₁-C₆ alkoxy, halo, —OH, and —CN. In some embodiments, Ring B is C₆-C₁₀ aryl or C₃-C₆ cycloalkyl, each of which is optionally substituted by 5 substituents selected from C₁-C₆ alkyl, C₁-C₆ haloalkyl, C₁-C₆ alkoxy, halo, —OH, and —CN. In some embodiments, Ring B is C₆-C₁₀ aryl or C₃-C₆ cycloalkyl, each of which is optionally substituted by 4 substituents selected from C₁-C₆ alkyl, C₁-C₆ haloalkyl, C₁-C₆ alkoxy, halo, —OH, and —CN. In some embodiments, Ring B is C₆-C₁₀ aryl or C₃-C₆ cycloalkyl, each of which is optionally substituted by 1-3 substituents selected from C₁-C₆ alkyl, C₁-C₆ haloalkyl, C₁-C₆ alkoxy, halo, —OH, and —CN. In some embodiments, Ring B is C₆-C₁₀ aryl or C₃-C₆ cycloalkyl, each of which is optionally substituted by 3 substituents selected from C₁-C₆ alkyl, C₁-C₆ haloalkyl, C₁-C₆ alkoxy, halo, —OH, and —CN. In some embodiments, Ring B is C₆-C₁₀ aryl and C₃-C₆ cycloalkyl, each of which is optionally substituted by 2 substituents selected from C₁-C₆ alkyl, C₁-C₆ haloalkyl, C₁-C₆ alkoxy, halo, —OH, and —CN. In some embodiments, Ring B is C₆-C₁₀ aryl or C₃-C₆ cycloalkyl, each of which is optionally substituted by 1 substituent selected from C₆-C₁₀ alkyl, C₁-C₆ haloalkyl, C₁-C₆ alkoxy, halo, —OH, and —CN. In some embodiments, Ring B is C₆-C₁₀ aryl or C₃-C₆ cycloalkyl, each of which is optionally substituted by 1 substituent selected from C₁-C₃ alkyl, C₁-C₃ haloalkyl, C₁-C₃ alkoxy, halo, —OH, and —CN. In some embodiments, Ring B is phenyl or cyclohexyl, each of which is optionally substituted by 1 substituent selected from C₁-C₃ alkyl, C₁-C₃ haloalkyl, C₁-C₃ alkoxy, halo, —OH, and —CN. In some embodiments, Ring B is phenyl or cyclohexyl, each of which is optionally substituted by 1 substituent selected from propyl, ethyl, methyl, CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, halo, —OH, and —CN. In some embodiments, Ring B is phenyl or cyclohexyl, each of which is optionally substituted by 1 substituent selected from methyl, —CF₃, —CHF₂, —CH₂F, —OCH₃, halo, —OH, and —CN. In some embodiments, Ring B is phenyl or cyclohexyl, each of which is optionally substituted by methyl. In some embodiments, Ring B is phenyl optionally substituted by methyl. In some embodiments, Ring B is cyclohexyl optionally substituted by methyl. In some embodiments, Ring B is phenyl substituted by methyl.

In some embodiments, Ring B is C₆-C₁₀ aryl optionally substituted by 1-5 substituents selected from C₁-C₆ alkyl, C₁-C₆ haloalkyl, C₁-C₆ alkoxy, halo, —OH, and —CN. In some embodiments, Ring B is C₆-C₁₀ aryl optionally substituted by 5 substituents selected from C₁-C₆ alkyl, C₁-C₆ haloalkyl, C₁-C₆ alkoxy, halo, —OH, and —CN. In some embodiments, Ring B is C₆-C₁₀ aryl optionally substituted by 4 substituents selected from C₁-C₆ alkyl, C₁-C₆ haloalkyl, C₁-C₆ alkoxy, halo, —OH, and —CN. In some embodiments, Ring B is C₆-C₁₀ aryl optionally substituted by 1-3 substituents selected from C₁-C₆ alkyl, C₁-C₆ haloalkyl, C₁-C₆ alkoxy, halo, —OH, and —CN. In some embodiments, Ring B is C₆-C₁₀ aryl optionally substituted by 3 substituents selected from C₁-C₆ alkyl, C₁-C₆ haloalkyl, C₁-C₆ alkoxy, halo, —OH, and —CN. In some embodiments, Ring B is C₆-C₁₀ aryl optionally substituted by 2 substituents selected from C₁-C₆ alkyl, C₁-C₆ haloalkyl, C₁-C₆ alkoxy, halo, —OH, and —CN. In some embodiments, Ring B is C₆-C₁₀ aryl optionally substituted by 1 substituent selected from C₆-C₁₀ alkyl, C₁-C₆ haloalkyl, C₁-C₆ alkoxy, halo, —OH, and —CN. In some embodiments, Ring B is C₆-C₁₀ aryl optionally substituted by 1 substituent selected from C₁-C₃ alkyl, C₁-C₃ haloalkyl, C₁-C₃ alkoxy, halo, —OH, and —CN. In some embodiments, Ring B is phenyl optionally substituted by 1 substituent selected from C₁-C₃ alkyl, C₁-C₃ haloalkyl, C₁-C₃ alkoxy, halo, —OH, and —CN. In some embodiments, Ring B is phenyl optionally substituted by 1 substituent selected from propyl, ethyl, methyl, CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, halo, —OH, and —CN. In some embodiments, Ring B is phenyl optionally substituted by 1 substituent selected from methyl, —CF₃, —CHF₂, —CH₂F, —OCH₃, halo, —OH, and —CN. In some embodiments, Ring B is phenyl optionally substituted by methyl. In some embodiments, Ring B is phenyl substituted by methyl.

In some embodiments, Ring B is C₃-C₆ cycloalkyl optionally substituted by 1-5 substituents selected from C₁-C₆ alkyl, C₁-C₆ haloalkyl, C₁-C₆ alkoxy, halo, —OH, and —CN. In some embodiments, Ring B is C₃-C₆ cycloalkyl optionally substituted by 5 substituents selected from C₁-C₆ alkyl, C₁-C₆ haloalkyl, C₁-C₆ alkoxy, halo, —OH, and —CN. In some embodiments, Ring B is C₃-C₆ cycloalkyl optionally substituted by 4 substituents selected from C₁-C₆ alkyl, C₁-C₆ haloalkyl, C₁-C₆ alkoxy, halo, —OH, and —CN. In some embodiments, Ring B is C₃-C₆ cycloalkyl optionally substituted by 1-3 substituents selected from C₁-C₆ alkyl, C₁-C₆ haloalkyl, C₁-C₆ alkoxy, halo, —OH, and —CN. In some embodiments, Ring B is C₃-C₆ cycloalkyl optionally substituted by 1 substituent selected from C₆-C₁₀ alkyl, C₁-C₆ haloalkyl, C₁-C₆ alkoxy, halo, —OH, and —CN. In some embodiments, Ring B is C₃-C₆ cycloalkyl optionally substituted by 3 substituents selected from C₁-C₆ alkyl, C₁-C₆ haloalkyl, C₁-C₆ alkoxy, halo, —OH, and —CN. In some embodiments, Ring B is C₃-C₆ cycloalkyl optionally substituted by 2 substituents selected from C₁-C₆ alkyl, C₁-C₆ haloalkyl, C₁-C₆ alkoxy, halo, —OH, and —CN. In some embodiments, Ring B is C₃-C₆ cycloalkyl optionally substituted by 1 substituent selected from C₁-C₃ alkyl, C₁-C₃ haloalkyl, C₁-C₃ alkoxy, halo, —OH, and —CN. In some embodiments, Ring B is cyclohexyl optionally substituted by 1 substituent selected from C₁-C₃ alkyl, C₁-C₃ haloalkyl, C₁-C₃ alkoxy, halo, —OH, and —CN. In some embodiments, Ring B is cyclohexyl optionally substituted by 1 substituent selected from propyl, ethyl, methyl, CH₂CH₂CF₃, —CH₂CH₂CHF₂, —CH₂CH₂CH₂F, —CH₂CF₃, —CH₂CHF₂, —CH₂CH₂F, —CF₃, —CHF₂, —CH₂F, —OCH₂CH₂CH₃, —OCH₂CH₃, —OCH₃, halo, —OH, and —CN. In some embodiments, Ring B is cyclohexyl optionally substituted by 1 substituent selected from methyl, —CF₃, —CHF₂, —CH₂F, —OCH₃, halo, —OH, and —CN. In some embodiments, Ring B is cyclohexyl, optionally substituted by methyl.

In some embodiments, Ring B is

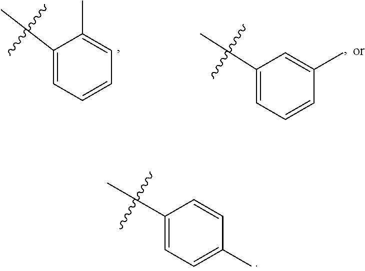

In some embodiments, Ring B is

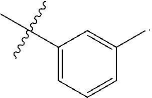 [SMILES: *c1cccc(C)c1]

In some embodiments, X is CH or N. In some embodiments, X is CH. In some embodiments, X is N.

In some embodiments, L is —O(C₁-C₆ alkylene)C(O)—, —O(C₁-C₆ alkylene)-C(O)NR¹(C₁-C₆ alkylene)C(O)—, —O(C₁-C₆ alkylene)C(O)NR¹(C₁-C₆ alkylene)-O—(C₁-C₆ alkylene)C(O)—, —O(C₁-C₆ alkylene)C(O)NR¹(C₁-C₆ alkylene)-, or —C(O)(6- to 11-membered spiro heterocyclene)-. In some embodiments, L is —O(C₁-C₃ alkylene)C(O)—, —O(C₁-C₃ alkylene)C(O)NR¹(C₁-C₃ alkylene)C(O)—, —O(C₁-C₃ alkylene)C(O)NR¹(C₁-C₃ alkylene)-O—(C₁-C₃ alkylene)C(O)—, —O(C₁-C₃ alkylene)C(O)NR¹(C₁-C₃ alkylene)-, or —C(O)(6- to 11-membered spiro heterocyclene)-.

In some embodiments, L is —O(C₁-C₆ alkylene)C(O)—. In some embodiments, L is —O(C₁-C₃ alkylene)C(O)—. In some embodiments, L is —OCH₂C(O)—, —OCH₂CH₂C(O)—, or —OCH₂CH₂CH₂C(O)—. In some embodiments, L is —OCH₂C(O)—.

In some embodiments, L is —O(C₁-C₆ alkylene)C(O)NR¹(C₁-C₆ alkylene)C(O)—. In some embodiments, L is —O(C₁-C₃ alkylene)C(O)NR¹(C₁-C₃ alkylene)C(O)—. In some embodiments, L is —O(C₁ alkylene)C(O)NR¹(C₁-C₃ alkylene)C(O)—. In some embodiments, R¹ is H. In some embodiments, R¹ is C₁-C₆ alkyl. In some embodiments, R¹ is C₁-C₃ alkyl. In some embodiments, R¹ is methyl, ethyl, or propyl. In some embodiments, L is —OCH₂C(O)NH(CH₂)C(O)—. In some embodiments, L is —OCH₂C(O)NH(CH₂CH₂)C(O)—. In some embodiments, L is —OCH₂C(O)NH(CH₂CH₂CH₂)C(O)—.

In some embodiments, L is —O(C₁-C₆ alkylene)C(O)NR¹(C₁-C₆ alkylene)-O—(C₁-C₆ alkylene)C(O)—. In some embodiments, L is —O(C₁-C₃ alkylene)C(O)NR¹(C₁-C₃ alkylene)-O—(C₁-C₃ alkylene)C(O)—. In some embodiments, L is —O(C₁ alkylene)C(O)NR¹(C₁-C₃ alkylene)-O—(C₁-C₃ alkylene)C(O)—. In some embodiments, R¹ is H. In some embodiments, R¹ is C₁-C₆ alkyl. In some embodiments, R¹ is C₁-C₃ alkyl. In some embodiments, R¹ is methyl, ethyl, or propyl. In some embodiments, L is —OCH₂C(O)NHCH₂—O—CH₂C(O)—. In some embodiments, L is —OCH₂C(O)NHCH₂—O—CH₂CH₂C(O)—. In some embodiments, L is —OCH₂C(O)NHCH₂—O—CH₂CH₂C(O)—. In some embodiments, L is —OCH₂C(O)NHCH₂—O—CH₂CH₂CH₂C(O)—. In some embodiments, L is —OCH₂C(O)NHCH₂CH₂—O—CH₂C(O)—. In some embodiments, L is —OCH₂C(O)NHCH₂CH₂—O—CH₂CH₂C(O)—. In some embodiments, L is —OCH₂C(O)NHCH₂CH₂—O—CH₂CH₂CH₂C(O)—. In some embodiments, L is —OCH₂C(O)NHCH₂CH₂CH₂—O—CH₂C(O)—. In some embodiments, L is —OCH₂C(O)NHCH₂CH₂CH₂—O—CH₂CH₂C(O)—. In some embodiments, L is —OCH₂C(O)NHCH₂CH₂CH₂—O—CH₂CH₂CH₂C(O)—.

In some embodiments, L is —O(C₁-C₆ alkylene)C(O)NR¹(C₁-C₆ alkylene)-. In some embodiments, L is —O(C₁-C₃ alkylene)C(O)NR¹(C₁-C₃ alkylene)-. In some embodiments, L is —O(C₁ alkylene)C(O)NR¹(C₁-C₃ alkylene)-. In some embodiments, R¹ is H. In some embodiments, R¹ is C₁-C₆ alkyl. In some embodiments, R¹ is C₁-C₃ alkyl. In some embodiments, R¹ is methyl, ethyl, or propyl. In some embodiments, L is —OCH₂C(O)NHCH₂—. In some embodiments, L is —OCH₂C(O)NHCH₂CH₂—. In some embodiments, L is —OCH₂C(O)NHCH₂CH₂CH₂—.

In some embodiments, L is —C(O)(6- to 11-membered spiro heterocyclene)-. In some embodiments, L is —C(O)(6-membered spiro heterocyclene)-. In some embodiments, L is —C(O)(7-membered spiro heterocyclene)-. In some embodiments, L is —C(O)(8-membered spiro heterocyclene)-. In some embodiments, L is —C(O)(9-membered spiro heterocyclene)-. In some embodiments, L is —C(O)(10-membered spiro heterocyclene)-. In some embodiments, L is —C(O)(11-membered spiro heterocyclene)-.

In some embodiments, L is

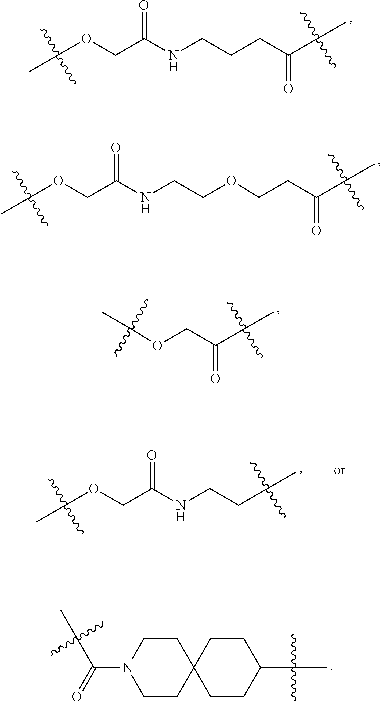 [SMILES: *OCC(=O)NCCCC(*)=O ; *OCC(=O)NCCOCCC(*)=O ; *OCC(*)=O ; *CCNC(=O)CO* ; *C(=O)N1CCC2(CCC(*)CC2)CC1]

In some embodiments, R^{2a} and R^{2b} are each H or are taken together to form an oxo group. In some embodiments, R^{2a} and R^{2b} are each H. In some embodiments, R^{2a} and R^{2b} are taken together to form an oxo group.

In some embodiments, the

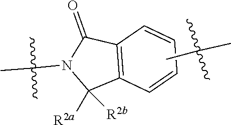

moiety of Formula (I) is

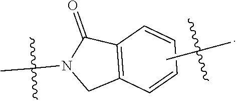

In some embodiments, the

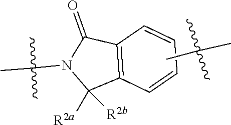

moiety of Formula (I) is

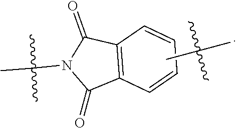

In some embodiments, the

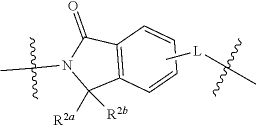

moiety of Formula (I) is

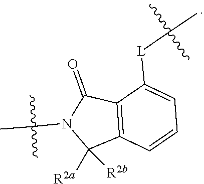

In some embodiments, the

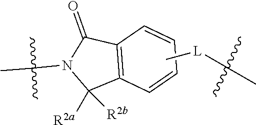

moiety of Formula (I) is

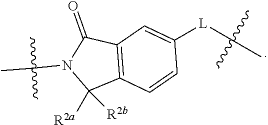

In some embodiments, the

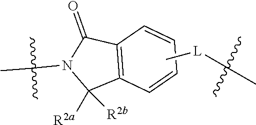

moiety of Formula (I) is

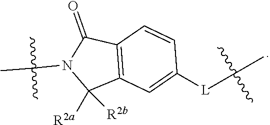

In some embodiments, the

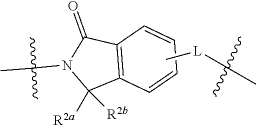

moiety of Formula (I) is

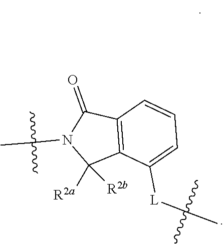

In some embodiments, the compound of Formula (I) is a compound of Formula (II):

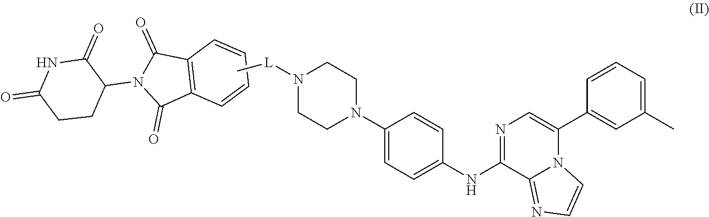

wherein L is as described for Formula (I).

In some embodiments, the compound of Formula (I) is a compound of Formula (II-A) or (II-B):

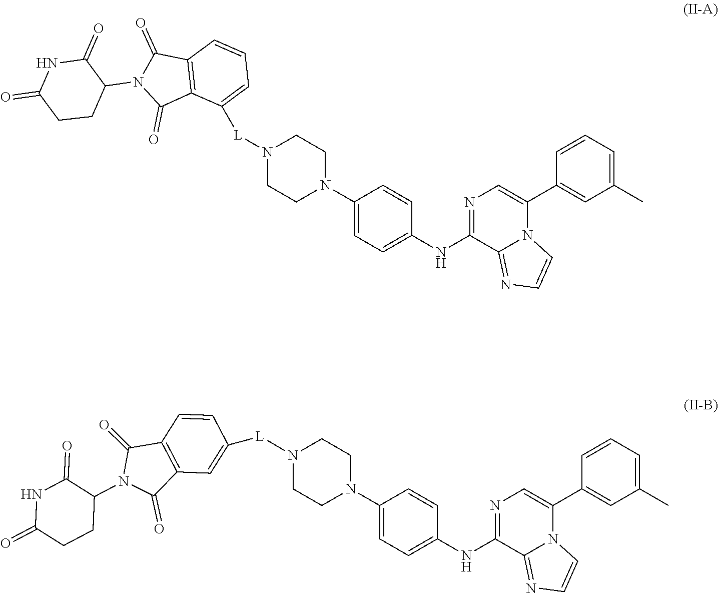

wherein L is as described for Formula (I).

In some embodiments, the compound of Formula (I) is a compound of Formula (IIIa) or (IIIb):

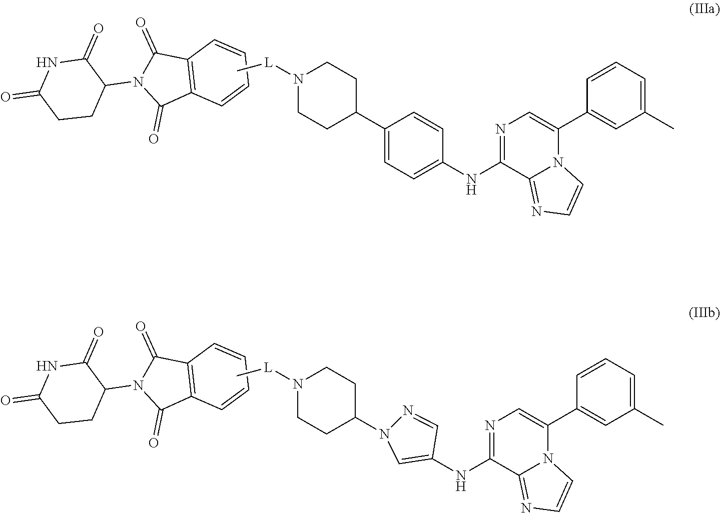

wherein L is as described for Formula (I).

In some embodiments, the compound of Formula (I) is a compound of Formula (III-A), (III-B), (III-C), or (III-D):

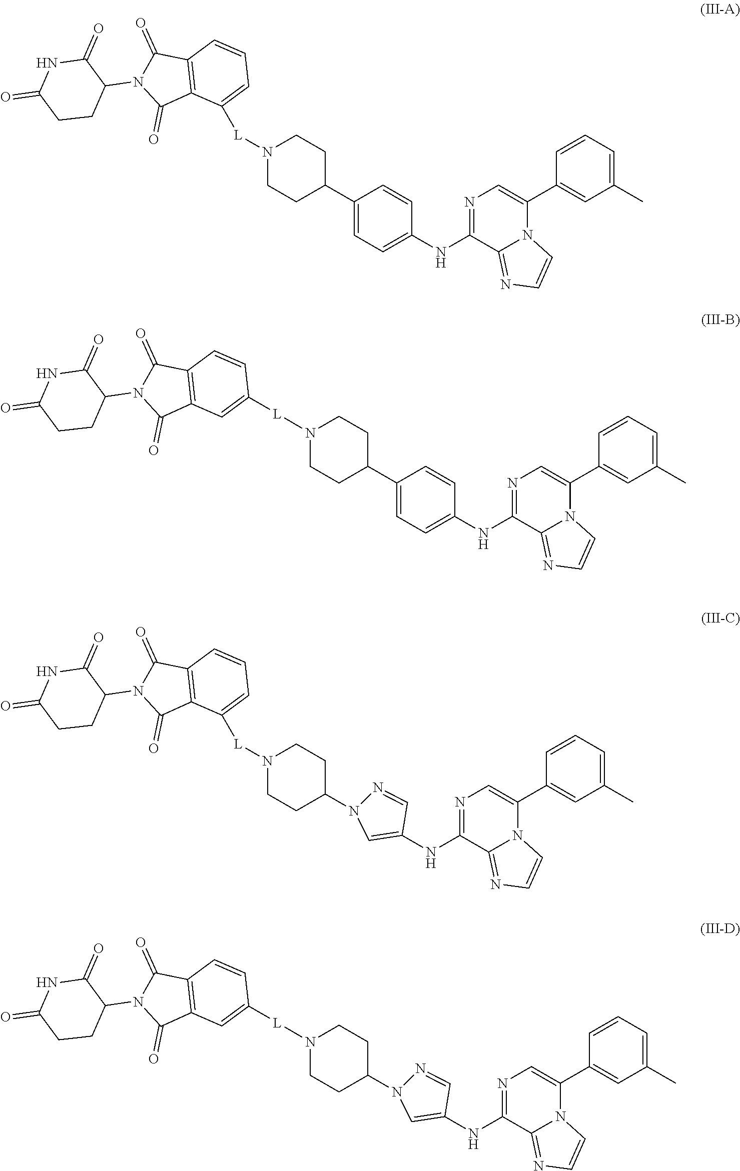

wherein L is as described for Formula (I).

In some embodiments, the compound of Formula (I) is a compound of Formula (IV):

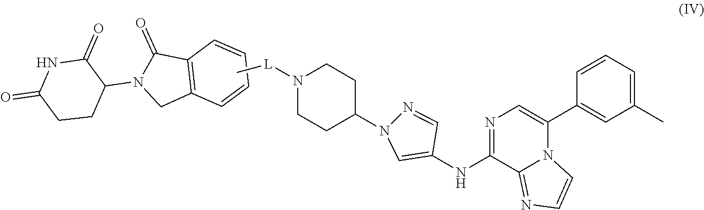

wherein L is as described for Formula (I).

In some embodiments, the compound of Formula (I) is a compound of Formula (IV-A) or (IV-B):

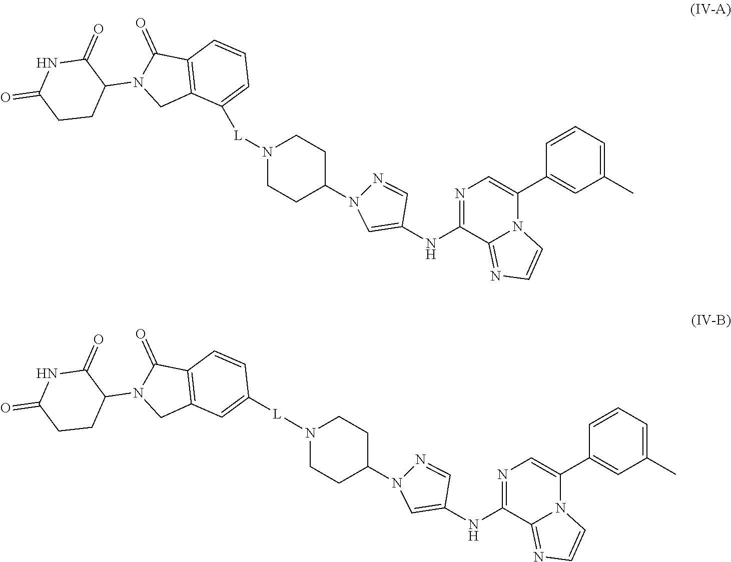

wherein L is as described for Formula (I).

In some embodiments, the compound of Formula (I) is a compound of Formula (V-A), (V-B), (V-C), (V-D), (V-E), or (V-F):

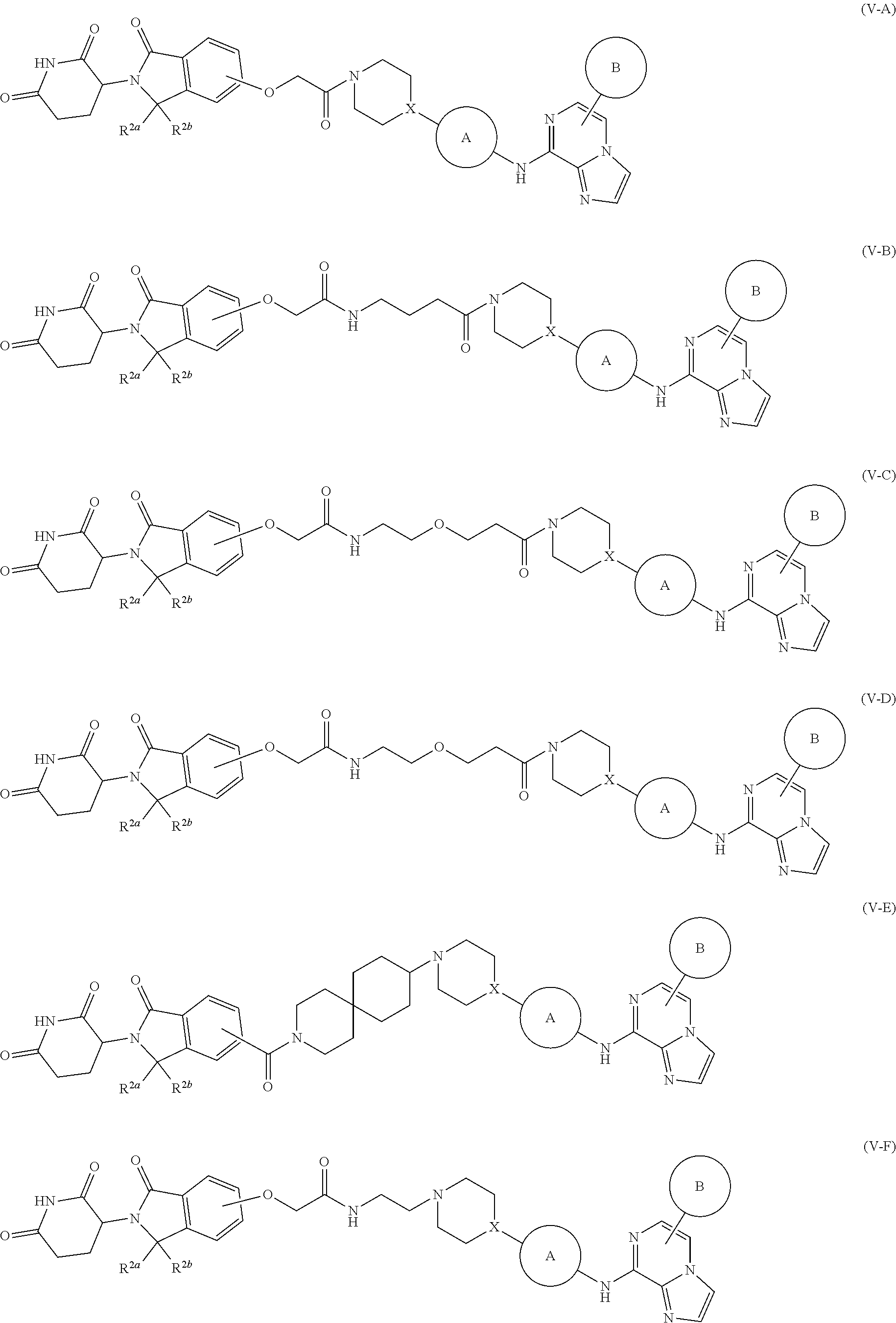

wherein Ring A, Ring B, R^{2a}, R^{2b}, and X are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of Ring A, Ring B, X, L, R^{2a}, and R^{2b} the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (II-A), (II-B), (IIIa), (IIIb), (III-A), (III-B), (III-C), (III-D), (IV), (IV-A), (V-B), (V-A), (V-B), (V-C, (V-D), (V-E), and (V-F), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | | |
|---|---|---|
| No. | Structure | Name |
| 1 | 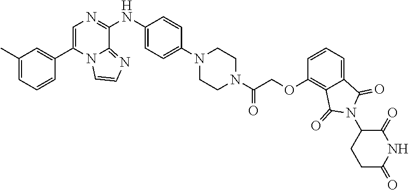 [SMILES: Cc1cccc(-c2cnc(Nc3ccc(N4CCN(C(=O)COc5cccc6c5C(=O)N(C5CCC(=O)NC5=O)C6=O)CC4)cc3)c3nccn23)c1] | 2-(2,6-Dioxopiperidin-3- yl)-4-(2-oxo-2-(4-(4-((5- (m-tolyl)imidazo[1,2- a]pyrazin-8- yl)amino)phenyl)piperazin- 1-yl)ethoxy)isoindoline- 1,3-dione |
| 2 | 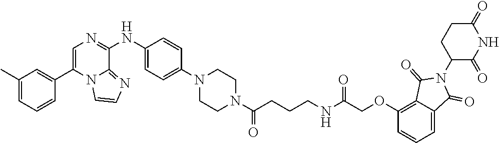 [SMILES: Cc1cccc(-c2cnc(Nc3ccc(N4CCN(C(=O)CCCNC(=O)COc5cccc6c5C(=O)N(C5CCC(=O)NC5=O)C6=O)CC4)cc3)c3nccn23)c1] | 2-((2-(2,6-Dioxopiperidin- 3-yl)-1,3-dioxoisoindolin- 4-yl)oxy)-N-(4-oxo-4-(4- (4-((5-(m- tolyl)imidazo[1,2- a]pyrazin-8- yl)amino)phenyl)piperazin- 1-yl)butyl)acetamide |
| 3 | 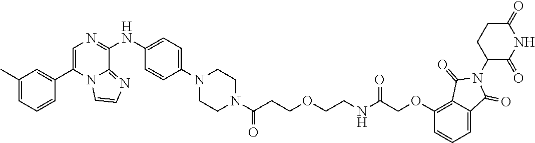 [SMILES: Cc1cccc(-c2cnc(Nc3ccc(N4CCN(C(=O)CCOCCNC(=O)COc5cccc6c5C(=O)N(C5CCC(=O)NC5=O)C6=O)CC4)cc3)c3nccn23)c1] | 2-((2-(2,6-Dioxopiperidin- 3-yl)-1,3-dioxoisoindolin- 4-yl)oxy)-N-(2-(3-oxo-3- (4-(4-((5-(m- tolyl)imidazo[1,2- a]pyrazin-8- yl)amino)phenyl)piperazin- 1- yl)propoxy)ethyl)acetamide |
| 4 | 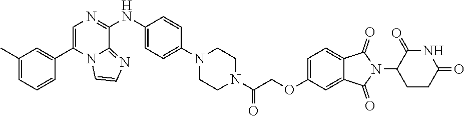 [SMILES: Cc1cccc(-c2cnc(Nc3ccc(N4CCN(C(=O)COc5ccc6c(c5)C(=O)N(C5CCC(=O)NC5=O)C6=O)CC4)cc3)c3nccn23)c1] | 2-(2,6-Dioxopiperidin-3- yl)-5-(2-oxo-2-(4-(4-((5- (m-tolyl)imidazo[1,2- a]pyrazin-8- yl)amino)phenyl)piperazin- 1-yl)ethoxy)isoindoline- 1,3-dione |
| 5 | 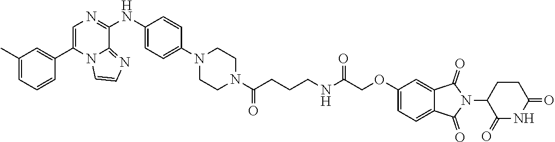 [SMILES: Cc1cccc(-c2cnc(Nc3ccc(N4CCN(C(=O)CCCNC(=O)COc5ccc6c(c5)C(=O)N(C5CCC(=O)NC5=O)C6=O)CC4)cc3)c3nccn23)c1] | 2-((2-(2,6-Dioxopiperidin- 3-yl)-1,3-dioxoisoindolin- 5-yl)oxy)-N-(4-oxo-4-(4- (4-((5-(m- tolyl)imidazo[1,2- a]pyrazin-8- yl)amino)phenyl)piperazin- 1-yl)butyl)acetamide |
| 6 | 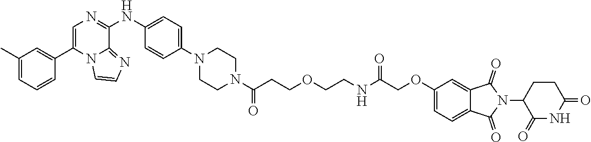 [SMILES: Cc1cccc(-c2cnc(Nc3ccc(N4CCN(C(=O)CCOCCNC(=O)COc5ccc6c(c5)C(=O)N(C5CCC(=O)NC5=O)C6=O)CC4)cc3)c3nccn23)c1] | 2-((2-(2,6-Dioxopiperidin- 3-yl)-1,3-dioxoisoindolin- 5-yl)oxy)-N-(2-(3-oxo-3- (4-(4-((5-(m- tolyl)imidazo[1,2- a]pyrazin-8- yl)amino)phenyl)piperazin- 1- yl)propoxy)ethyl)acetamide |
| 7 | 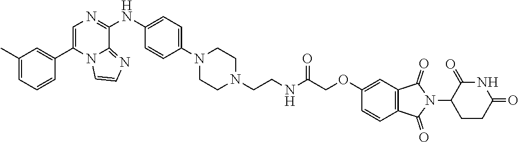 [SMILES: Cc1cccc(-c2cnc(Nc3ccc(N4CCN(CCNC(=O)COc5ccc6c(c5)C(=O)N(C5CCC(=O)NC5=O)C6=O)CC4)cc3)c3nccn23)c1] | 2-((2-(2,6-Dioxopiperidin- 3-yl)-1,3-dioxoisoindolin- 5-yl)oxy)-N-(2-(4-(4-((5- (m-tolyl)imidazo[1,2- a]pyrazin-8- yl)amino)phenyl)piperazin- 1-yl)ethyl)acetamide |
| 8 | 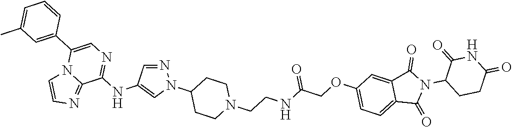 [SMILES: Cc1cccc(-c2cnc(Nc3cnn(C4CCN(CCNC(=O)COc5ccc6c(c5)C(=O)N(C5CCC(=O)NC5=O)C6=O)CC4)c3)c3nccn23)c1] | 2-((2-(2,6-Dioxopiperidin- 3-yl)-1,3-dioxoisoindolin- 5-yl)oxy)-N-(2-(4-(4-((5- (m-tolyl)imidazo[1,2- a]pyrazin-8-yl)amino)-1H- pyrazol-1-yl)piperidin-1- yl)ethyl)acetamide |
| 9 | 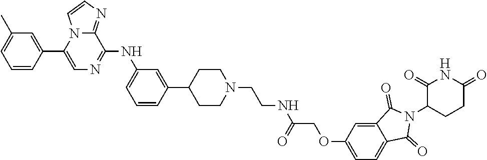 [SMILES: Cc1cccc(-c2cnc(Nc3cccc(C4CCN(CCNC(=O)COc5ccc6c(c5)C(=O)N(C5CCC(=O)NC5=O)C6=O)CC4)c3)c3nccn23)c1] | 2-((2-(2,6-Dioxopiperidin- 3-yl)-1,3-dioxoisoindolin- 5-yl)oxy)-N-(2-(4-(3-((5- (m-tolyl)imidazo[1,2- a]pyrazin-8- yl)amino)phenyl)piperidin- 1-yl)ethyl)acetamide |
| 10 | 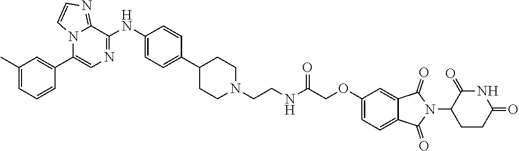 [SMILES: Cc1cccc(-c2cnc(Nc3ccc(C4CCN(CCNC(=O)COc5ccc6c(c5)C(=O)N(C5CCC(=O)NC5=O)C6=O)CC4)cc3)c3nccn23)c1] | 2-((2-(2,6-Dioxopiperidin- 3-yl)-1,3-dioxoisoindolin- 5-yl)oxy)-N-(2-(4-(4-((5- (m-tolyl)imidazo[1,2- a]pyrazin-8- yl)amino)phenyl)piperidin- 1-yl)ethyl)acetamide |
| 11 | 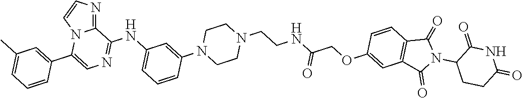 [SMILES: Cc1cccc(-c2cnc(Nc3cccc(N4CCN(CCNC(=O)COc5ccc6c(c5)C(=O)N(C5CCC(=O)NC5=O)C6=O)CC4)c3)c3nccn23)c1] | 2-((2-(2,6-Dioxopiperidin- 3-yl)-1,3-dioxoisoindolin- 5-yl)oxy)-N-(2-(4-(3-((5- (m-tolyl)imidazo[1,2- a]pyrazin-8- yl)amino)phenyl)piperazin- 1-yl)ethyl)acetamide |
| 12 | 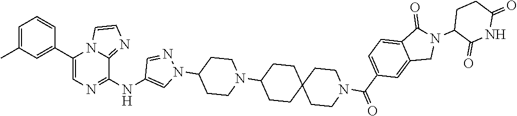 [SMILES: Cc1cccc(-c2cnc(Nc3cnn(C4CCN(C5CCC6(CC5)CCN(C(=O)c5ccc7c(c5)CN(C5CCC(=O)NC5=O)C7=O)CC6)CC4)c3)c3nccn23)c1] | 3-(1-oxo-5-(9-(4-(4-((5- (m-tolyl)imidazo[1,2- a] pyrazin-8-yl)amino)-1H- pyrazol-1-yl)piperidin-1- yl)-3- azaspiro[5.5]undecane-3- carbonyl)isoindolin-2- yl)piperidine-2,6-dione |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Schemes 1-3, as well as in the examples set forth herein. It should be noted that one skilled in the art would know how to modify the procedures set forth in the illustrative schemes and examples to arrive at the desired products.

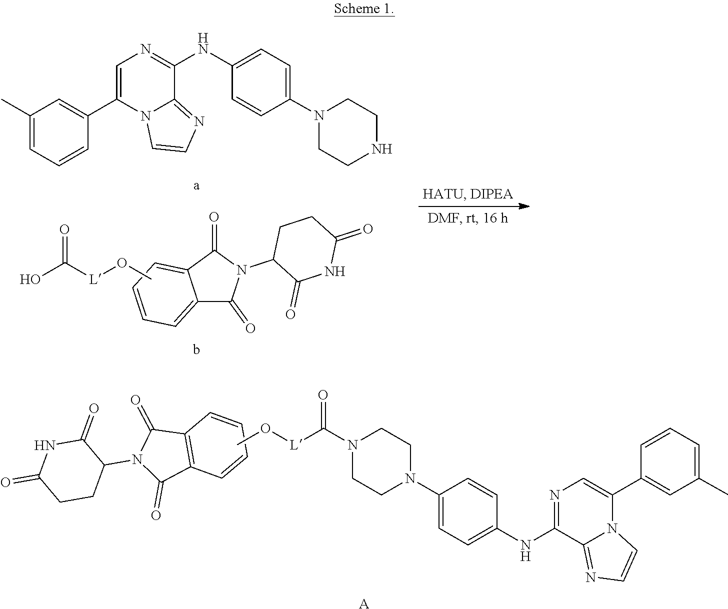

wherein L′ is —(C₁-C₆ alkylene)-, —(C₁-C₆ alkylene)C(O)NR¹(C₁-C₆ alkylene)-, or —(C₁-C₆ alkylene)C(O)NR¹(C₁-C₆ alkylene)-O—(C₁-C₆ alkylene)-; and Ring A is as described for Formula (I).

As outlined in Scheme 1, compounds of general formula A can be synthesized by reacting intermediate a and intermediate b with various amide coupling reagents to generate compounds of general formula A.

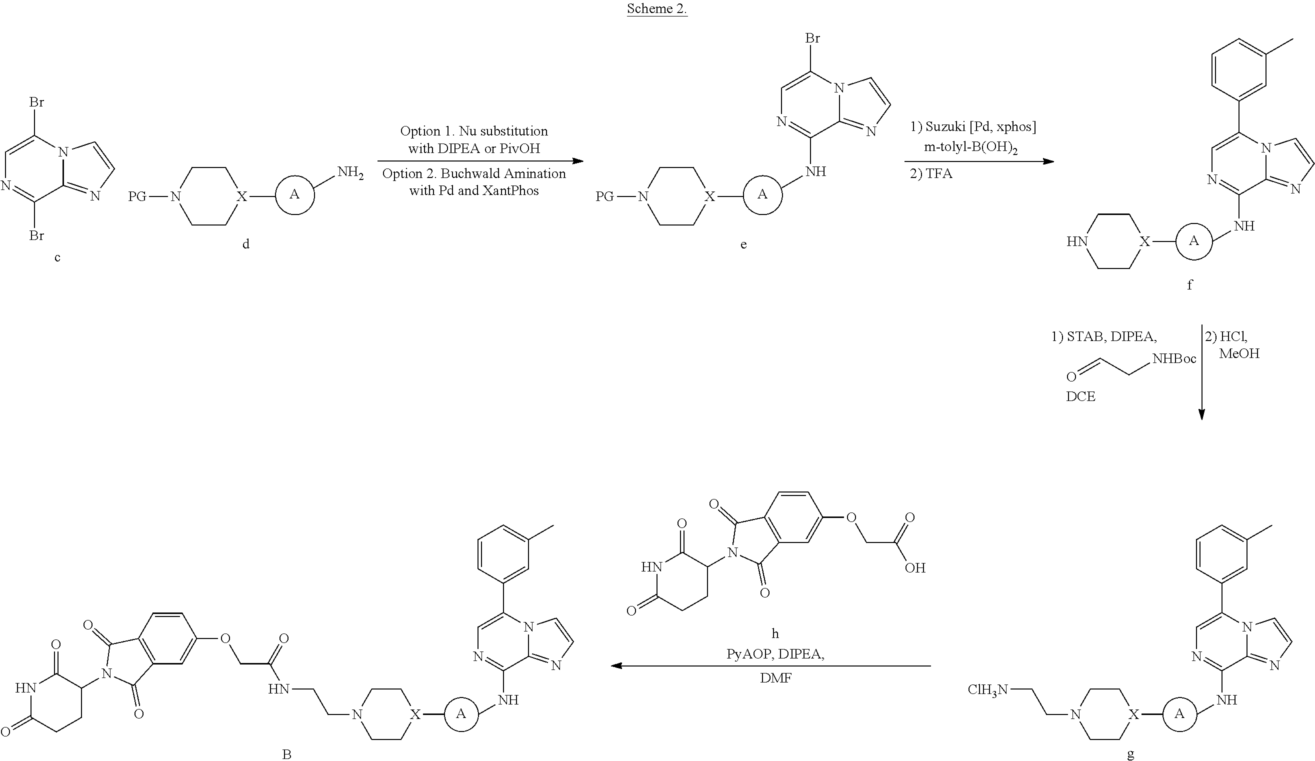

wherein PG is a protecting group; and X and Ring A is as described for Formula (I).

As outlined in Scheme 2, compounds of general formula B that contain various substituted heteroaryls or aryls can be synthesized by coupling intermediate c and intermediate d to generate intermediate e. For example, a Buchwald-Hartwig amination reaction using intermediate c and intermediate d, catalyzed by a palladium salt and a suitable ligand, can be used to prepare the bromo-imidazopyrazine intermediate e. A subsequent Suzuki cross-coupling reaction with m-tolyl boronic acid and routine Boc-group deprotection (for example, using TFA) affords intermediate f. Intermediate g can be generated by reductive amination of tert-butyl N-(2-oxoethyl)carbamate with intermediate f in the presence of a reducing agent, such as STAB, and a base, such as DIPEA, followed by Boc-deprotection with HCl (or TFA). Lastly, amide coupling of intermediate g with intermediate h through the use of an amide coupling reagent such as PyAOP with DIPEA affords compounds of general formula B.

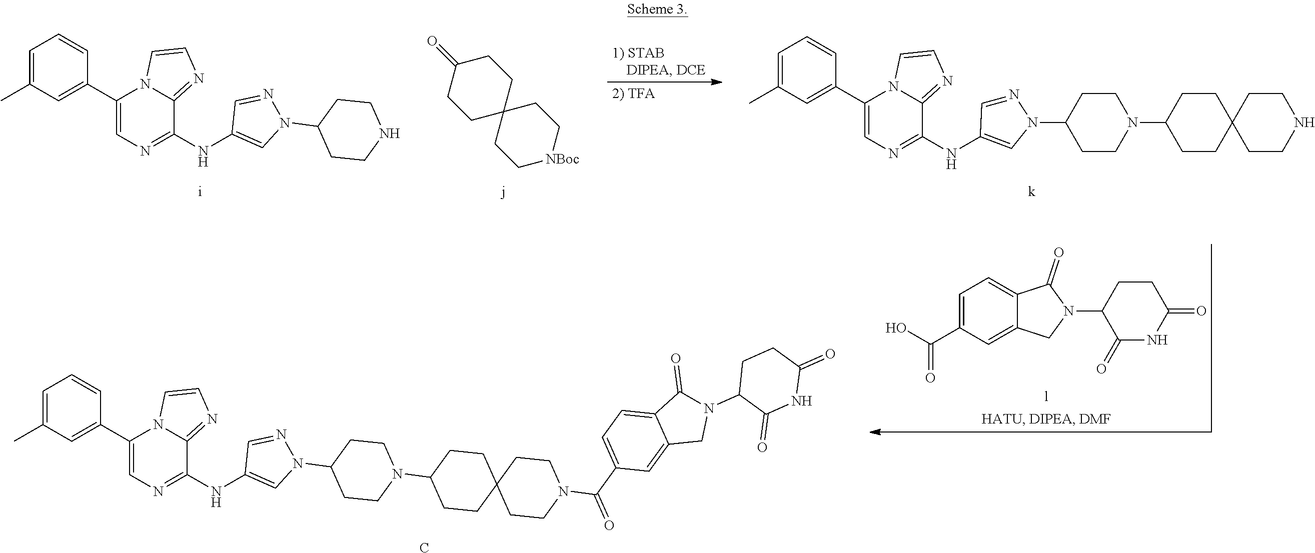

The compound of formula C, containing a spirocyclic linker, can be synthesized through a route analogous to the one shown in Scheme 2. As outlined in Scheme 3, this compound, which is shown as a representative example of compounds bearing spirocyclic linkers, can be synthesized by utilizing a spirocyclic ketone (intermediate j) rather than a straight chain aldehyde. Reductive amination of intermediate i with ketone-bearing intermediate j and a reducing agent, such as STAB, and base, followed by Boc-deprotection affords spirocyclic intermediate k. In a final step, coupling of the carboxylic acid of intermediate 1 with the secondary amine of intermediate k using an amide coupling reagent, such as HATU, affords the compound of formula C.

### Methods of Use

Embodiments of the present disclosure provide a method for modulating IRAK3 in a subject in need thereof, the method comprising administering to the subject an effective amount of a compound of Formula (I). Modulation (e.g., inhibition or activation) of IRAK3 can be assessed and demonstrated by a wide variety of ways known in the art. Kits and commercially available assays can be utilized for determining whether and to what degree IRAK3 has been modulated (e.g., inhibited or activated).

In one aspect, provided herein is a method of modulating IRAK3 comprising contacting IRAK3 with an effective amount of a compound of Formula (I) or any embodiment or variation thereof. In some embodiments, the compound of Formula (I) inhibits IRAK3. In some embodiments, the compound of Formula (I) causes degradation of IRAK3.

In some embodiments, provided herein is a method for targeting IRAK3 for degradation comprising contacting IRAK3 with an effective amount of a compound of Formula (I) or any embodiment or variation thereof.

In some embodiments, a compound of Formula (I) modulates the activity of IRAK3 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of IRAK3 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK3 in a subject in need thereof, the method comprising administering to the subject an effective amount of a compound of Formula (I). Degradation of IRAK3 can be assessed and demonstrated by a wide variety of ways known in the art. Kits and commercially available assays, including cell-based assays, can be utilized for determining whether and to what degree IRAK3 has been degraded.

In one aspect, provided herein is a method of degrading IRAK3 comprising contacting IRAK3 with an effective amount of a compound of Formula (I) or any embodiment or variation thereof. In some embodiments, the compound of Formula (I) partially degrades IRAK3. In some embodiments, the compound of Formula (I) fully degrades IRAK3.

In some embodiments, a compound of Formula (I) degrades IRAK3 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades IRAK3 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating cancer in a subject in need thereof, comprising administering to the subject an effective amount of a compound of Formula (I). In some embodiments, provided herein is a method for preventing cancer in a subject in need thereof, comprising administering to the subject an effective amount of a compound of Formula (I). Non-limiting examples of cancer include bladder cancer, breast cancer, esophageal cancer, colon cancer, head and neck cancer, kidney cancer, lung cancer, pancreatic cancer, prostate cancer, melanoma, and gastric cancer.

In some embodiments, administering a compound of Formula (I) to a subject that is predisposed to cancer prevents the subject from developing any symptoms of the cancer (such as tumor growth or metastasis). In some embodiments, administering a compound of Formula (I) to a subject that does not yet display symptoms of cancer prevents the subject from developing any symptoms of the cancer. In some embodiments, administering a compound of Formula (I) to a subject in need thereof diminishes the extent of the cancer in the subject. In some embodiments, administering a compound of Formula (I) to a subject in need thereof stabilizes the cancer (prevents or delays the worsening of the cancer). In some embodiments, administering a compound of Formula (I) to a subject in need thereof delays the occurrence or recurrence of the cancer. In some embodiments, administering a compound of Formula (I) to a subject in need thereof slows the progression of the cancer. In some embodiments, administering a compound of Formula (I) to a subject in need thereof provides a partial remission of the cancer. In some embodiments, administering a compound of Formula (I) to a subject in need thereof provides a total remission of the cancer. In some embodiments, administering a compound of Formula (I) to a subject in need thereof decreases the dose of one or more other medications required to treat the cancer. In some embodiments, administering a compound of Formula (I) to a subject in need thereof enhances the effect of another medication used to treat the cancer. In some embodiments, administering a compound of Formula (I) to a subject in need thereof delays the progression of the cancer. In some embodiments, administering a compound of Formula (I) to a subject in need thereof increases the quality of life of the subject having cancer. In some embodiments, administering a compound of Formula (I) to a subject in need thereof prolongs survival of a subject having cancer.

In one aspect, provided herein is method of preventing a subject that is predisposed to cancer from developing cancer, the method comprising administering a compound of Formula (I) to the subject.

In some aspects, provided herein is a method of diminishing the extent of cancer in a subject, the method comprising administering a compound of Formula (I) to the subject. In some embodiments, provided herein is a method of stabilizing cancer in a subject, the method comprising administering a compound of Formula (I) to the subject. In some embodiments, the method prevents the worsening of the cancer.

In another aspect, provided herein is a method of delaying the occurrence or recurrence of cancer in a subject, the method comprising administering a compound of Formula (I) to the subject.

In some embodiments, provided herein is a method of slowing the progression of cancer in a subject, the method comprising administering a compound of Formula (I) to the subject. In some embodiments, the method provides a partial remission of the cancer. In some embodiments, the method provides a total remission of the cancer.

In further aspects, provided herein is a method of decreasing the dose of one or more other medications required to treat cancer in a subject, the method comprising administering a compound of Formula (I) to the subject. In some embodiments, provided herein is a method of enhancing the effect of another medication used to treat cancer in a subject, the method comprising administering a compound of Formula (I) to the subject.

Also provided here is a method of delaying the progression of cancer in a subject, the method comprising administering a compound of Formula (I) to the subject. In some embodiments, the method increases the quality of life of the subject having cancer. In some embodiments, the method prolongs survival of the subject having cancer.

In some embodiments, compounds of Formula (I) are useful for treating a cancer selected from bladder cancer, breast cancer, esophageal cancer, colon cancer, head and neck cancer, kidney cancer, lung cancer, pancreatic cancer, prostate cancer, melanoma, and gastric cancer.

In some embodiments, provided herein is a method of enhancing immunity in a subject receiving a vaccine, comprising administering to the subject an effective amount of a compound of Formula (I). In some embodiments, the compound of Formula (I) is administered to the subject prior to the administration of a vaccine. In some embodiments, the compound of Formula (I) is administered to the subject simultaneously to the administration of a vaccine. In some embodiments, the compound of Formula (I) is administered to the subject following the administration of a vaccine. In some embodiments, the compound of Formula (I) is formulated as a component of the vaccine. In some embodiments, the compound of Formula (I) is formulated separately from the vaccine.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I) administered will depend on such factors as the solubility of the active component, the formulation used and the route of administration.

In some embodiments, a compound of Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

In a particular embodiment, provided herein are unit dosage formulations comprising about 0.1 mg or 100 mg of a compound of Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (I).

A compound of Formula (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### Exemplary Embodiments

The present disclosure is further described by the following embodiments. The features of each of the embodiments are combinable with any of the other embodiments where appropriate and practical.

Embodiment 1. A compound of Formula (I):

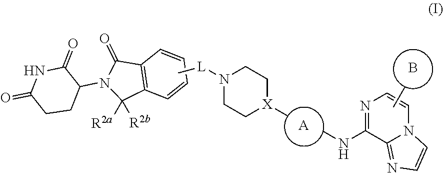

or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
Ring B is C₆-C₁₀ aryl or C₃-C₆ cycloalkyl, each of which is optionally substituted by 1-5 substituents selected from C₁-C₆ alkyl, C₁-C₆ haloalkyl, C₁-C₆ alkoxy, halo, —OH, and —CN;
X is CH or N;
L is —O(C₁-C₆ alkylene)C(O)—, —O(C₁-C₆ alkylene)C(O)NR¹(C₁-C₆ alkylene)C(O)—, —O(C₁-C₆ alkylene)C(O)NR¹(C₁-C₆ alkylene)-O—(C₁-C₆ alkylene)C(O)—, —O(C₁-C₆ alkylene)C(O)NR¹(C₁-C₆ alkylene)-, or —C(O)(6- to 11-membered spiro heterocyclene)-;
R¹ is H or C₁-C₆ alkyl; and
R^{2a} and R^{2b} are each H or are taken together to form an oxo group.

Embodiment 2 The compound of embodiment 1, or a pharmaceutically acceptable salt thereof, wherein:
Ring A is C₆-C₁₀ aryl.

Embodiment 3. The compound of embodiment 1, or a pharmaceutically acceptable salt thereof, wherein:
Ring A is 5- to 6-membered heteroaryl, wherein the heteroaryl contains 1-3 nitrogens.

Embodiment 4. The compound of embodiment 1 or 2, or a pharmaceutically acceptable salt thereof, wherein:
Ring A is, or

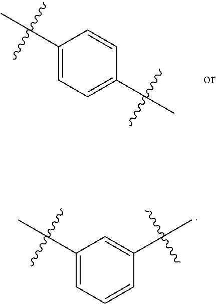 [SMILES: *c1ccc(*)cc1 ; *c1cccc(*)c1]

Embodiment 5. The compound of embodiment 1 or 3, or a pharmaceutically acceptable salt thereof, wherein:
Ring A is

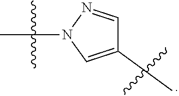 [SMILES: *c1cnn(*)c1]

Embodiment 6. The compound of any one of embodiments 1-5, or a pharmaceutically acceptable salt thereof, wherein Ring B is C₆-C₁₀ aryl optionally substituted by 1-5 substituents selected from C₁-C₆ alkyl.

Embodiment 7. The compound of embodiment 6, or a pharmaceutically acceptable salt thereof, wherein Ring B is C₆-C₁₀ aryl optionally substituted by 1-3 substituents selected from C₁-C₃ alkyl.

Embodiment 8. The compound of any one of embodiments 1-7, or a pharmaceutically acceptable salt thereof, wherein:
Ring B is

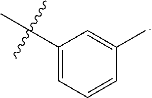 [SMILES: *c1cccc(C)c1]

Embodiment 9. The compound of any one of embodiments 1-8, or a pharmaceutically acceptable salt thereof, wherein X is CH.

Embodiment 10. The compound of any one of embodiments 1-8, or a pharmaceutically acceptable salt thereof, wherein X is N.

Embodiment 11. The compound of any one of embodiments 1-10, or a pharmaceutically acceptable salt thereof, wherein:
L is —O(C₁-C₃ alkylene)C(O)—, —O(C₁-C₃ alkylene)C(O)NR¹(C₁-C₃ alkylene)C(O)—, —O(C₁-C₃ alkylene)C(O)NR¹(C₁-C₃ alkylene)-O—(C₁-C₃ alkylene)C(O)—, —O(C₁-C₃ alkylene)C(O)NR¹(C₁-C₃ alkylene)-, or —C(O)(10- to 11-membered spiro heterocyclene)-; and
R¹ is H.

Embodiment 12. The compound of any one of embodiments 1-11, or a pharmaceutically acceptable salt thereof, wherein L is:

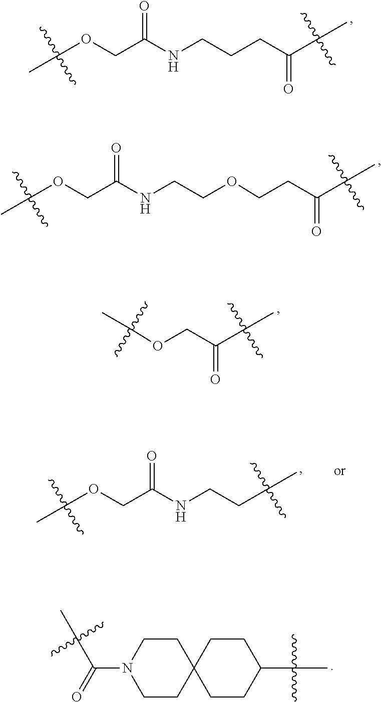 [SMILES: *OCC(=O)NCCCC(*)=O ; *OCC(=O)NCCOCCC(*)=O ; *OCC(*)=O ; *CCNC(=O)CO* ; *C(=O)N1CCC2(CCC(*)CC2)CC1]

Embodiment 13. The compound of any one of embodiments 1-12, or a pharmaceutically acceptable salt thereof, wherein R^{2a} and R^{2b} are each H.

Embodiment 14. The compound of any one of embodiments 1-12, or a pharmaceutically acceptable salt thereof, wherein R^{2a} and R^{2b} are taken together to form an oxo group.

Embodiment 15. The compound of any one of embodiments 1, 2, 4, 6-8, 10, 12, or 14, or a pharmaceutically acceptable salt thereof, wherein the compound is Formula (II):

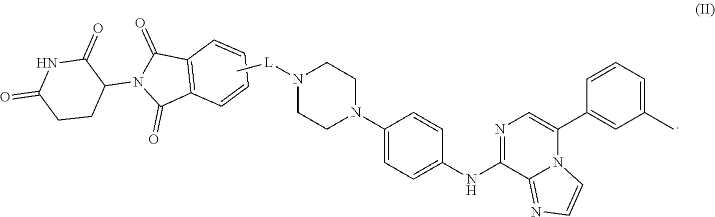

Embodiment 16. The compound of any one of embodiments 1, 2, 4, 6-9, 11, 12, or 14, or a pharmaceutically acceptable salt thereof, wherein the compound is Formula (IIIa):

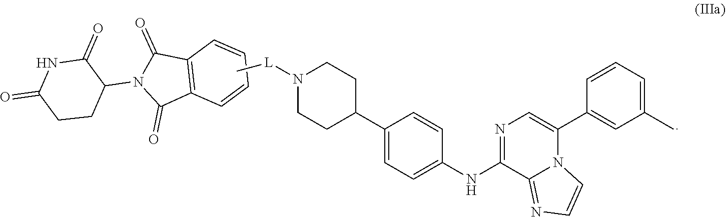

Embodiment 17. The compound of any one of embodiments 1, 3, 5, 6-9, 11, 12, or 14, or a pharmaceutically acceptable salt thereof, wherein the compound is Formula (IIIb):

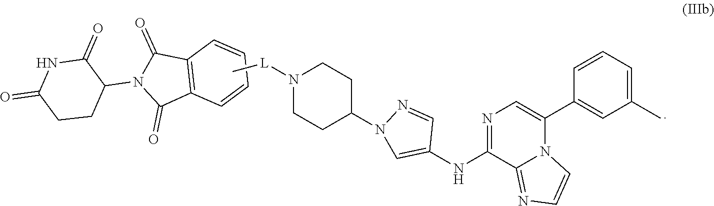

Embodiment 18. The compound of any one of embodiments 1, 2, 4, 9, 10, or 13-15, or a pharmaceutically acceptable salt thereof, wherein the compound is Formula (IV).

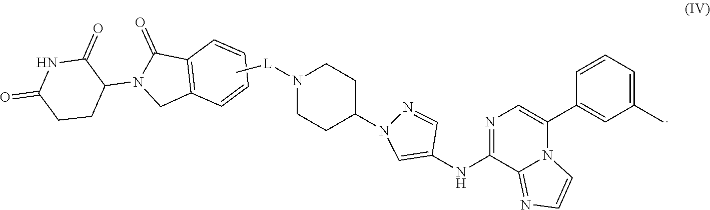

Embodiment 19. A compound selected from the compounds of Table 1 or a pharmaceutically acceptable salt thereof.

Embodiment 20. A pharmaceutical composition comprising the compound of any one of embodiments 1-19, or a pharmaceutically acceptable salt thereof, and a pharmaceutically acceptable excipient.

Embodiment 21. A method of modulating Interleukin-1 Receptor-Associated Kinase 3 (IRAK3) comprising contacting IRAK3 with an effective amount of the compound of any one of embodiments 1-19, or a pharmaceutically acceptable salt thereof, or the pharmaceutical composition of embodiment 20.

Embodiment 22. A method of treating cancer in a subject in need thereof, comprising administering to the subject an effective amount of the compound of any one of embodiments 1-19, or a pharmaceutically acceptable salt thereof, or the pharmaceutical composition of embodiment 20.

Embodiment 23. The method of embodiment 22, wherein the cancer is selected from bladder cancer, breast cancer, esophageal cancer, colon cancer, head and neck cancer, kidney cancer, lung cancer, pancreatic cancer, prostate cancer, melanoma, and gastric cancer.

Embodiment 24. A method of enhancing immunity in a subject receiving a vaccine, comprising administering to the subject an effective amount of the compound of any one of embodiments 1-19, or a pharmaceutically acceptable salt thereof, or the pharmaceutical composition of embodiment 20.

Embodiment 25. The method of embodiment 24, wherein the subject is administered the vaccine prior to, concurrently with, or after administration of the compound of any one of embodiments 1-19, or a pharmaceutically acceptable salt thereof, or the pharmaceutical composition of embodiment 20.

### Examples

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

| | |
|---|---|
| Boc | tert-butyloxycarbonyl |
| Bu | butyl |
| CBz | benzyloxycarbonyl |
| CH₂Cl₂ | dichloromethane |
| Cs₂CO₃ | cesium carbonate |
| DCM | dichloromethane |
| DIEA/DIPEA/Hünig's Base | diisopropylethylamine |
| DMF | dimethyl formamide |
| DMSO | dimethyl sulfoxide |
| Et | ethyl |
| EtOAc | ethyl acetate |
| EtOH | ethanol |
| FA | formic acid |
| H₂ | hydrogen (gas) |
| H₂O | water |
| H₂SO₄ | sulfuric acid |
| HATU | O-(7-azabenzotriazol-1-yl)-N,N,N′,N′-tetramethyluronium |
| | hexafluorophosphate |
| HCl | hydrochloric acid |
| i-Pr | isopropyl |
| K₂CO₃ | potassium carbonate |
| K₃PO₄ | potassium phosphate |
| Me | methyl |
| MeCN | acetonitrile |
| MeOH | methanol |
| N₂ | nitrogen (gas) |
| Na₂SO₄ | sodium sulfate |
| NaBH(OAc)₃ / STAB | sodium triacetoxyborohydride |
| NaBH₃CN | sodium cyanoborohydride |
| NaHCO₃ | sodium bicarbonate |
| NH₄Cl | ammonium chloride |
| Pd(PPh₃)₄ | palladium tetrakis |
| Pd/C | palladium on carbon |
| Pd₂(dba)₃ | tris(dibenzylideneacetone)dipalladium(0) |
| PdCl₂(dppf) | [1,1′-bis(diphenylphosphino)-ferrocene]dichloropalladium(II) |
| pin | pinacolato |
| PivOH | pivalic acid |
| SiO₂ | silica oxide |
| t-BuOK | Potassium tert-butoxide |
| TEA | triethylamine |
| TFA | trifluoroacetic acid |
| THF | tetrahydrofuran |
| Xantphos | 4,5-Bis(diphenylphosphino)-9,9-dimethylxanthene |
| Xphos | 2-dicyclohexylphosphino-2′,4′,6′-triisopropylbiphenyl |
| Xphos Pd G₃ | 2-dicyclohexylphosphino-2′,4′,6′-triisopropyl-1,1′-biphenyl)[2- |
| | (2′-amino-1,1′-biphenyl)]palladium(II) methanesulfonate |

### Synthetic Examples

### Analytical Methods

LC/MS Method 1. Column: Luna C18(2) 50×3 mm, 3 um. Temperature: 45° C., Flow: 2 mL/min, Run time: 2 min. Mobile phase conditions: Initial 95% H₂O 0.1% FA/5% MeCN 0.1% FA, linear gradient to 95% MeCN 0.1% FA over 1 min then hold for 1 minute at 95% CH₃CN 0.1% FA.]

LC/MS Method 2. Waters Acquity UPLC system. Column: Acquity UPLC BEH C18 1.7 mM (2.1×50 mm). Modifer: Formic Acid. Mobile Phase: Water-0.1% formic acid (A) and Acetonitrile-0.1% formic acid (B). Flow rate: 0.8 mL/min. Gradient: 5% B to 95% B in 1.5 min, holding at 95% B for 0.5 min, 95% B to 5% B in 0.1 min. Detector 1 UV 214 nm and 254 nm

LC/MS Method 3. SunFire C18 75×4.6 mm, 3.5 um. Temperature: 45° C., Flow: 1.5 mL/min, Run time: 6 min. Mobile phase conditions: Initial 95% H2O+0.1% FA/5% MeCN+0.1% FA then linear gradient to 95% MeCN for 4 min then hold for 2 min at 95% MeCN.

LC/MS Method 4. Luna C18(2) 50×3 mm, 3 um. Temperature: 45° C., Flow: 1.5 mL/min, Run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95% MeCN 0.1% FA.

### Example I-1. Preparation of 2-((2-(2,6-Dioxopiperidin-3-yl)-1,3-dioxoisoindolin-4-yl)oxy)acetic acid (I-1)

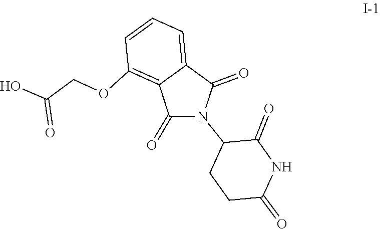 [SMILES: O=C(O)COc1cccc2c1C(=O)N(C1CCC(=O)NC1=O)C2=O]

Commercially available from a multitude of vendors.

### Example I-2. Preparation of 4-(2-((2-(2,6-dioxopiperidin-3-yl)-1,3-dioxoisoindolin-4-yl)oxy)acetamido)butanoic acid (I-2)

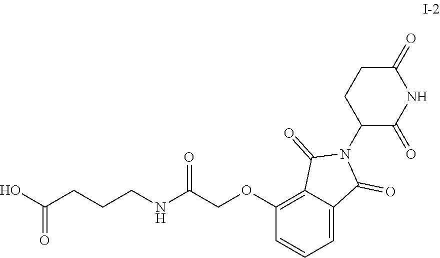 [SMILES: O=C(O)CCCNC(=O)COc1cccc2c1C(=O)N(C1CCC(=O)NC1=O)C2=O]

Commercially available from Astatech, Inc. and Matrix Scientific at the time of writing.

### Example I-3. Preparation of 3-(2-(2-((2-(2,6-dioxopiperidin-3-yl)-1,3-dioxoisoindolin-4-yl)oxy)acetamido)ethoxy)propanoic acid (I-3)

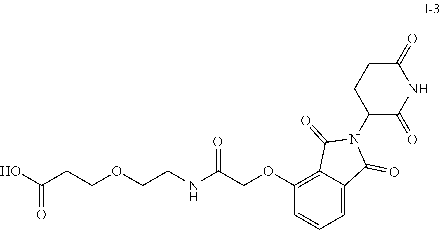 [SMILES: O=C(O)CCOCCNC(=O)COc1cccc2c1C(=O)N(C1CCC(=O)NC1=O)C2=O]

Previously reported in: Chessum et al., Journal of Medicinal Chemistry, 2018, 61 (3), 918-933.

### Example I-4. Preparation of 2-((2-(2,6-dioxopiperidin-3-yl)-1,3-dioxoisoindolin-5-yl)oxy)acetic acid (I-4)

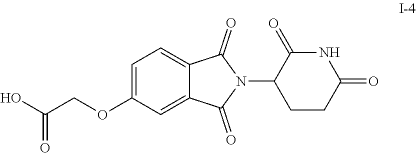 [SMILES: O=C(O)COc1ccc2c(c1)C(=O)N(C1CCC(=O)NC1=O)C2=O]

Previously reported in: Bricelj et al., ACS Med. Chem. Lett., 2021, 12 (11), 1733-1738.

### Example I-5. Preparation of 4-(2-((2-(2,6-dioxopiperidin-3-yl)-1,3-dioxoisoindolin-5-yl)oxy)acetamido)butanoic acid (I-5)

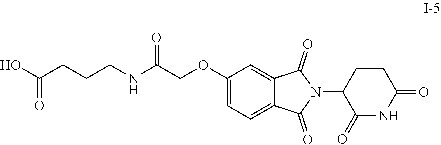 [SMILES: O=C(O)CCCNC(=O)COc1ccc2c(c1)C(=O)N(C1CCC(=O)NC1=O)C2=O]

Prepared analogously to I-6 (below). LC/MS Method 2: MS (ESI) [M+H]⁺ 418, rt: 1.37 min.

### Example I-6. Preparation of 3-(2-(2-((2-(2,6-dioxopiperidin-3-yl)-1,3-dioxoisoindolin-5-yl)oxy)acetamido)ethoxy)propanoic acid (I-6)

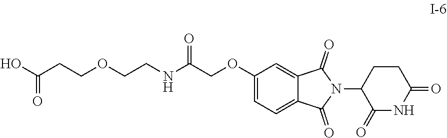 [SMILES: O=C(O)CCOCCNC(=O)COc1ccc2c(c1)C(=O)N(C1CCC(=O)NC1=O)C2=O]

tert-Butyl 3-(2-(2-((2-(2,6-dioxopiperidin-3-yl)-1,3-dioxoisoindolin-5-yl)oxy)acetamido)ethoxy)propanoate. To a solution of 2-((2-(2,6-dioxopiperidin-3-yl)-1,3-dioxoisoindolin-5-yl)oxy)acetic acid (1.8 g, 5.42 mmol) and HATU (2.472 g, 6.50 mmol) in DMF (20 mL) was added DIEA (2.365 mL, 13.54 mmol), followed by tert-butyl 3-(2-aminoethoxy)propanoate (1.025 g, 5.42 mmol). The reaction mixture was stirred at RT for 1 h. The reaction mixture was then concentrated was purified by normal phase column chromatography (0 to 100% EtOAc in hexanes, followed by 0 to 20% MeOH in DCM), affording the product, tert-butyl 3-(2-(2-((2-(2,6-dioxopiperidin-3-yl)-1,3-dioxoisoindolin-5-yl)oxy)acetamido)ethoxy)propanoate (1.4 g, 3.34 mmol, 62% yield).

3-(2-(2-((2-(2,6-Dioxopiperidin-3-yl)-1,3-dioxoisoindolin-5-yl)oxy)acetamido)ethoxy)propanoic acid. To a solution of tert-butyl 3-(2-(2-((2-(2,6-dioxopiperidin-3-yl)-1,3-dioxoisoindolin-5-yl)oxy)acetamido)ethoxy)propanoate (200 mg, 0.40 mmol) in DCM (4 mL) was added TFA (2 mL) at room temperature. The reaction mixture was stirred at room temperature for 2 h. The reaction was then concentrated, and purified by preparative HPLC, affording the product, 3-(2-(2-((2-(2,6-dioxopiperidin-3-yl)-1,3-dioxoisoindolin-5-yl)oxy)acetamido)ethoxy)propanoic acid (110 mg, 61% yield) as a white solid. LC/MS Method 2: MS (ESI) [M+H]⁺ 448, rt: 1.45 min.

### Example I-7. Preparation of 2-(2,6-dioxopiperidin-3-yl)-1-oxoisoindoline-5-carboxylic acid (I-7)

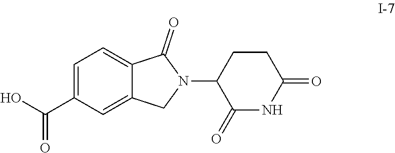 [SMILES: O=C1CCC(N2Cc3cc(C(=O)O)ccc3C2=O)C(=O)N1]

Commercially available from Sigma Aldrich and Enamine at the time of writing.

### Example I-8. Preparation of N-(4-(piperazin-1-yl)phenyl)-5-(m-tolyl)imidazo[1,2-a]pyrazin-8-amine (I-8)

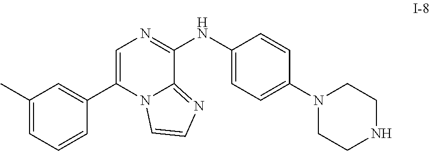 [SMILES: Cc1cccc(-c2cnc(Nc3ccc(N4CCNCC4)cc3)c3nccn23)c1]

tert-Butyl 4-(4-((5-bromoimidazo[1,2-a]pyrazin-8-yl)amino)phenyl)piperazine-1-carboxylate. tert-butyl 4-(4-aminophenyl)piperazine-1-carboxylate (604.3 mg, 2.18 mmol) and DIPEA (0.38 mL, 2.18 mmol) were added to a solution of 5,8-dibromoimidazo[1,2-a]pyrazine (402.2 mg, 1.45 mmol) in ethanol (2.69 mL). The reaction was heated to 80° C. and stirred overnight. LCMS showed that the reaction went to 94% conversion and the reaction was stopped. Ethanol was removed in vacuo and the product was taken up in DCM. The resulting solution was washed with water, dried over MgSO₄, and concentrated in vacuo. The residue was purified by reverse phase column chromatography (5% to 100% MeOH in water with 0.1% formic acid), affording the desired product, tert-butyl 4-(4-((5-bromoimidazo[1,2-a]pyrazin-8-yl)amino)phenyl)piperazine-1-carboxylate (443 mg, 0.935 mmol, 64% yield). LC/MS Method 1: MS (ESI) [M]⁺ 473.2, rt: 1.479 min.

tert-Butyl 4-(4-((5-(m-tolyl)imidazo[1,2-a]pyrazin-8-yl)amino)phenyl)piperazine-1-carboxylate. In a microwave vial, a mixture of 2 M aq. Na₂CO₃ (1.01 mL, 2.03 mmol) and m-tolylboronic acid (91.91 mg, 0.680 mmol) in 1,4-dioxane (5 mL) and tert-butyl 4-[4-[(5-bromoimidazo[1,2-a]pyrazin-8-yl)amino]phenyl]piperazine-1-carboxylate (320 mg, 0.680 mmol) was flushed with N₂ for 10 min, then Pd(Ph₃P)₄ (78.12 mg, 0.070 mmol) was added and the suspension was flushed for another 10 min with nitrogen. The reaction was heated to 100° C. and irradiated in a microwave reactor for 1 h. The reaction was then partitioned between EtOAc/H₂O and extracted with EtOAc (2×). The organic layer was then washed with H₂O, brine, dried over Na₂SO₄. The reaction was then concentrated directly onto silica gel and purified by normal phase chromatography (30% to 100% EtOAc in heptane), affording tert-butyl 4-(4-((5-(m-tolyl)imidazo[1,2-a]pyrazin-8-yl)amino)phenyl)piperazine-1-carboxylate (80% pure, 198 mg, 0.327 mmol, 48% yield), as an orange solid. LC/MS Method 1: MS (ESI) [M+H]⁺ 485.4, rt: 1.504 min.

N-(4-(Piperazin-1-yl)phenyl)-5-(m-tolyl)imidazo[1,2-a]pyrazin-8-amine. A RT solution of tert-butyl 4-[4-[[5-(m-tolyl)imidazo[1,2-a]pyrazin-8-yl]amino]phenyl]piperazine-1-carboxylate (580 mg, 1.2 mmol) in DCM (5 mL) was treated with TFA (3.0 mL, 11.76 mmol). The reaction was allowed to stir at for 4 h. The reaction mixture was concentrated to dryness, taken up in DCM and washed with sat. aq. NaHCO₃ (1×). The aqueous layer was then back-extracted with DCM (2×) and organic layers were combined and concentrated. The resulting residue was purified by reverse phase column chromatography (5% to 100% MeCN in water with 0.1% formic acid) affording the product, N-(4-(piperazin-1-yl)phenyl)-5-(m-tolyl)imidazo[1,2-a]pyrazin-8-amine (256.6 mg, 0.666 mmol, 56% yield), as an orange solid after lyophilization. MS (ESI) [M+H]⁺=385.2. ¹H NMR (400 MHz, DMSO-d₆) δ ppm 2.42 (s, 3H), 3.23 (br s, 4H), 3.31-3.59 (m, 4H), 7.10 (d, J=8.8 Hz, 2H), 7.38-7.43 (m, 1H), 7.44-7.60 (m, 4H), 7.79 (br d, J=7.6 Hz, 2H), 8.08-8.16 (m, 2H), 9.34 (br s, 2H), 10.93 (br s, 1H).

### Example I-9. N-(1-(Piperidin-4-yl)-1H-pyrazol-4-yl)-5-(m-tolyl)imidazo[1,2-a]pyrazin-8-amine (I-9)

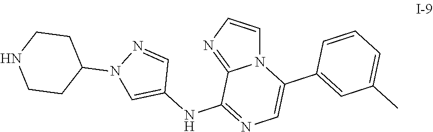 [SMILES: Cc1cccc(-c2cnc(Nc3cnn(C4CCNCC4)c3)c3nccn23)c1]

tert-Butyl 4-(4-((5-bromoimidazo[1,2-a]pyrazin-8-yl)amino)-1H-pyrazol-1-yl)piperidine-1-carboxylate. 5,8-Dibromoimidazo[1,2-a]pyrazine (500 mg, 1.8 mmol), tert-butyl 4-(4-aminopyrazol-1-yl)piperidine-1-carboxylate (500 mg, 1.05 equiv.) and pivalic acid (1.8 g, 10 equiv.) was added to a 2.0-5.0 mL microwave tube equipped with a stir bar. The tube was then sealed with a septum and heated to 100° C. under microwave irradiation for 1 h. LCMS analysis of the reaction mixture revealed full conversion of the starting material to the desired product (˜10% boc cleavage occurred). The reaction mixture was then diluted with EtOAc and transferred to a separatory funnel containing sat aq. NaHCO₃. The organic layer was then removed, and the aqueous layer was extracted twice with EtOAc. The combined organic layers were dried over sodium sulfate, filtered, and concentrated. The reaction mixture was then purified by reverse phase column chromatography (25% to 100% MeCN in water with 0.1% TFA), affording the product, tert-butyl 4-(4-((5-bromoimidazo[1,2-a]pyrazin-8-yl)amino)-1H-pyrazol-1-yl)piperidine-1-carboxylate (300 mg, 0.642 mmol, 35% yield), as a tan solid. LC/MS Method 2: MS (ESI) [M]⁺ 462.3, rt: 1.66 min.

N-(1-(Piperidin-4-yl)-1H-pyrazol-4-yl)-5-(m-tolyl)imidazo[1,2-a]pyrazin-8-amine. A 0.5-2 mL microwave reaction tube equipped with stir bar was charged with tert-butyl 4-[4-[(5-bromoimidazo[1,2-a]pyrazin-8-yl)amino]pyrazol-1-yl]piperidine-1-carboxylate (200 mg, 0.43 mmol), m-tolyl boronic acid (120 mg, 2.0 equiv.), Xphos (15.5 mg, 7.5 mol %) and XPhos-Pd-G3 (27.5 mg, 7.5 mol %). The reaction vessel was then sealed with a septum and purged with nitrogen. 1,4-Dioxane (2.2 mL, 0.2 M) and 2 M aqueous sodium carbonate (650 μL, 3 equiv.) were then added to the reaction vessel via syringe. The reaction vessel was then sealed with a septa cap and then mixed by vortex. The vessel was then heated at 135° C. under microwave irradiation for 45 min. After this time, the reaction was then allowed to cool to 22° C. and filtered through a pad of celite. The filter cake was then washed with EtOAc. LCMS analysis of the crude reaction mixture revealed full conversion of the starting material to the desired product. The reaction mixture was then purified by reverse phase column chromatography (25% to 100% MeCN in water with 0.1% TFA). The fractions were then concentrated and immediately dissolved in 1:1 DCM/TFA and allowed to stir at room temperature for 1 h, LCMS analysis revealed full deprotection to the desired product. The reaction mixture was again purified by reverse phase column chromatography (15% to 100% MeCN in water with 0.1% TFA), affording the product, N-(1-(piperidin-4-yl)-1H-pyrazol-4-yl)-5-(m-tolyl)imidazo[1,2-a]pyrazin-8-amine (141 mg, 0.379 mmol, 88% yield), as a light-yellow solid after lyophilization. MS (ESI) [M+H]⁺ 374.5. ¹H NMR (400 MHz, methanol-d₄) δ ppm 2.27-2.41 (m, 4H), 2.45 (s, 3H), 3.19-3.29 (m, 2H), 3.59 (br d, J=13.2 Hz, 2H), 4.60 (tt, J=10.1, 4.8 Hz, 1H), 7.31 (s, 1H), 7.39-7.51 (m, 4H), 7.81 (s, 2H), 7.94 (s, 1H), 8.24 (s, 1H).

### Library Procedure for Examples S1-6

Six 2-dram round bottom vials were charged with 0.5 mL of N, N-dimethylformamide (DMF) was first charged to the vials. To each vial, a 0.25 M solution of the corresponding intermediate (I-1 through I-6, 100 μL, 25 μmol) was added followed by a 0.25 M solution of HATU (100 μL, 50 μmol). A 0.25 M solution of N-(4-(piperazin-1-yl)phenyl)-5-(m-tolyl)imidazo[1,2-a]pyrazin-8-amine (120 μL, 30 μmol) was then added to each vial, followed by the addition of DIPEA (50 μL). The vials were then capped and allowed to shake at 25° C. for 16 hr. After removing the solvent by a Genevac EZ-2, the residues were re-dissolved with 1.0-2.0 mL of DMSO. The solutions were then purified by reverse phase prep-HPLC on a Waters Automated Purification System using a single quadruple mass detector (SQD2, Waters); a 19×100 mm XBridge™ C-18 prep column from Waters corporation. In each case, separation of library compounds was accomplished with a focused gradient from 10% to 90% CH₃CN in H₂O (containing v v 0.1% formic acid) at a flow rate of 20 mL/min. The resulting fractions were then concentrated on a Genevac EZ-2, affording the products as off-white solids (as formate salts).

### Example S1. 2-(2,6-Dioxopiperidin-3-yl)-4-(2-oxo-2-(4-(4-((5-(m-tolyl)imidazo[1,2-a]pyrazin-8-yl)amino)phenyl)piperazin-1-yl)ethoxy)isoindoline-1,3-dione (1)

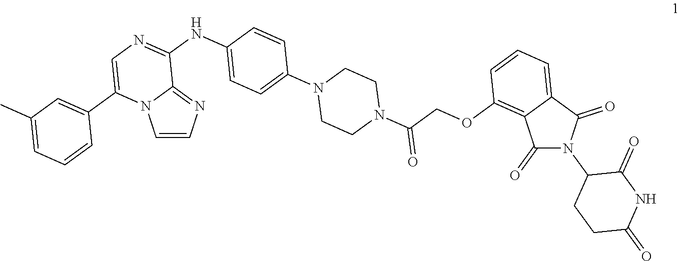 [SMILES: Cc1cccc(-c2cnc(Nc3ccc(N4CCN(C(=O)COc5cccc6c5C(=O)N(C5CCC(=O)NC5=O)C6=O)CC4)cc3)c3nccn23)c1]

Compound (1) was prepared by the general library procedure described above. LC/MS Method 2: MS (ESI) [M+H]⁺ 699.3, rt: 1.54 min.

### Example S2. 2-((2-(2,6-Dioxopiperidin-3-yl)-1,3-dioxoisoindolin-4-yl)oxy)-N-(4-oxo-4-(4-(4-((5-(m-tolyl)imidazo[1,2-a]pyrazin-8-yl)amino)phenyl)piperazin-1-yl)butyl)acetamide (2)

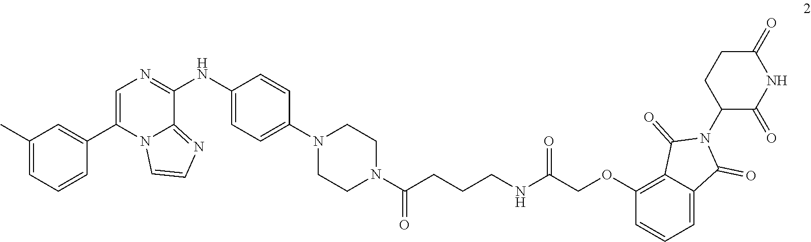 [SMILES: Cc1cccc(-c2cnc(Nc3ccc(N4CCN(C(=O)CCCNC(=O)COc5cccc6c5C(=O)N(C5CCC(=O)NC5=O)C6=O)CC4)cc3)c3nccn23)c1]

Compound (2) was prepared by the general library procedure described above. LC/MS Method 2: MS (ESI) [M+H]⁺ 784.4, rt: 1.48 min.

### Example S3. 2-((2-(2,6-Dioxopiperidin-3-yl)-1,3-dioxoisoindolin-4-yl)oxy)-N-(2-(3-oxo-3-(4-(4-((5-(m-tolyl)imidazo[1,2-a]pyrazin-8-yl)amino)phenyl)piperazin-1-yl)propoxy)ethyl)acetamide (3)

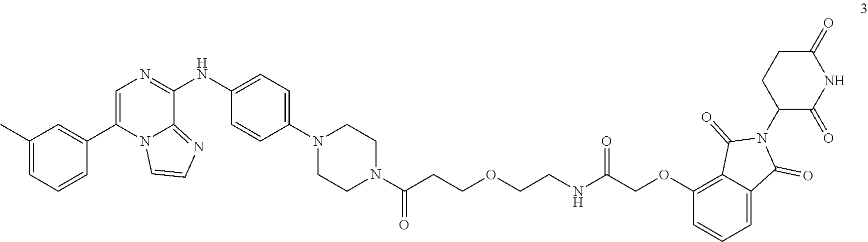 [SMILES: Cc1cccc(-c2cnc(Nc3ccc(N4CCN(C(=O)CCOCCNC(=O)COc5cccc6c5C(=O)N(C5CCC(=O)NC5=O)C6=O)CC4)cc3)c3nccn23)c1]

Compound (3) was prepared by the general library procedure described above. LC/MS Method 2: MS (ESI) [M+H]⁺ 814.5, rt: 1.47 min.

### Example S4. 2-(2,6-Dioxopiperidin-3-yl)-5-(2-oxo-2-(4-(4-((5-(m-tolyl)imidazo[1,2-a]pyrazin-8-yl)amino)phenyl)piperazin-1-yl)ethoxy)isoindoline-1,3-dione (4)

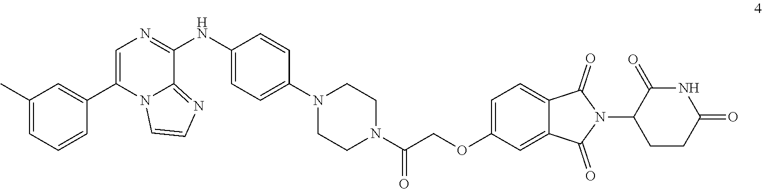 [SMILES: Cc1cccc(-c2cnc(Nc3ccc(N4CCN(C(=O)COc5ccc6c(c5)C(=O)N(C5CCC(=O)NC5=O)C6=O)CC4)cc3)c3nccn23)c1]

Compound (4) was prepared by the general library procedure described above. LC/MS Method 2: MS (ESI) [M+H]⁺ 699.3, rt: 1.55 min.

### Example S5. 2-((2-(2,6-Dioxopiperidin-3-yl)-1,3-dioxoisoindolin-5-yl)oxy)-N-(4-oxo-4-(4-(4-((5-(m-tolyl)imidazo[1,2-a]pyrazin-8-yl)amino)phenyl)piperazin-1-yl)butyl)acetamide (5)

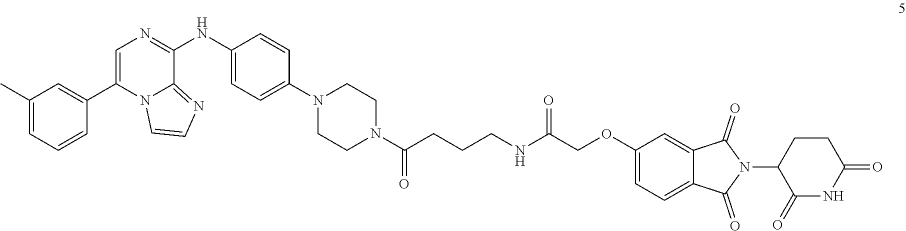 [SMILES: Cc1cccc(-c2cnc(Nc3ccc(N4CCN(C(=O)CCCNC(=O)COc5ccc6c(c5)C(=O)N(C5CCC(=O)NC5=O)C6=O)CC4)cc3)c3nccn23)c1]

Compound (5) was prepared by the general library procedure described above. LC/MS Method 2: MS (ESI) [M+H]⁺ 784.3, rt: 1.48 min.

### Example S6. 2-((2-(2,6-Dioxopiperidin-3-yl)-1,3-dioxoisoindolin-5-yl)oxy)-N-(2-(3-oxo-3-(4-(4-((5-(m-tolyl)imidazo[1,2-a]pyrazin-8-yl)amino)phenyl)piperazin-1-yl)propoxy)ethyl)acetamide (6)

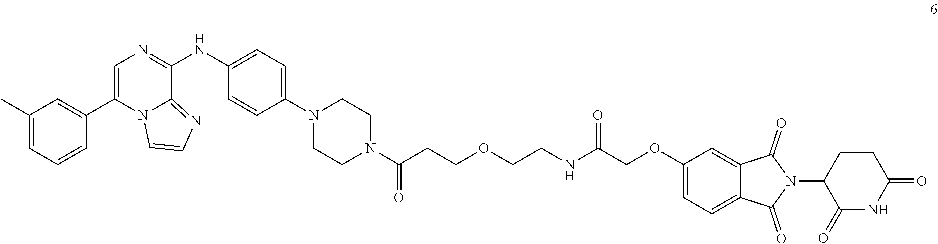 [SMILES: Cc1cccc(-c2cnc(Nc3ccc(N4CCN(C(=O)CCOCCNC(=O)COc5ccc6c(c5)C(=O)N(C5CCC(=O)NC5=O)C6=O)CC4)cc3)c3nccn23)c1]

Compound (6) was prepared by the general library procedure described above. LC/MS Method 2: MS (ESI) [M+H]⁺ 814.4, rt: 1.47 min.

### Example S7. 2-((2-(2,6-Dioxopiperidin-3-yl)-1,3-dioxoisoindolin-5-yl)oxy)-N-(2-(4-(4-((5-(m-tolyl)imidazo[1,2-a]pyrazin-8-yl)amino)phenyl)piperazin-1-yl)ethyl)acetamide (7)

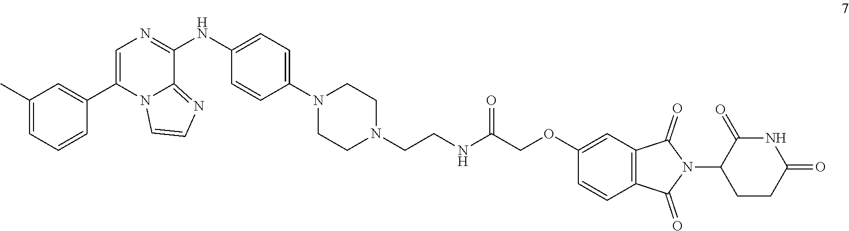 [SMILES: Cc1cccc(-c2cnc(Nc3ccc(N4CCN(CCNC(=O)COc5ccc6c(c5)C(=O)N(C5CCC(=O)NC5=O)C6=O)CC4)cc3)c3nccn23)c1]

tert-Butyl (2-(4-(4-((5-(m-tolyl)imidazo[1,2-a]pyrazin-8-yl)amino)phenyl)piperazin-1-yl)ethyl)carbamate. To a solution of N-(4-(piperazin-1-yl)phenyl)-5-(m-tolyl)imidazo[1,2-a]pyrazin-8-amine (43.0 mg, 0.10 mmol) in DMF (510 μL) were added tert-butyl N-(2-bromoethyl)carbamate (23.0 mg, 0.10 mmol) and DIPEA (270 μL, 1.53 mmol) at room temperature. The reaction was monitored by LCMS. After 72 h, LCMS showed around 50% conversion towards the desired product, an additional 0.5 equiv. of tert-butyl N-(2-bromoethyl)carbamate was added and the mixture stirred at room temperature. After 96 h, LCMS showed around 75% conversion towards the desired product, an additional 0.5 equiv. of tert-butyl N-(2-bromoethyl)carbamate was added and the mixture stirred at room temperature. After 128 h, LCMS showed around 85% conversion towards the desired product, an additional 0.5 equiv. of tert-butyl N-(2-bromoethyl)carbamate was added and the mixture stirred at 35° C. After 152 h of stirring, the reaction was stopped and directly purified by reverse phase flash chromatography (5% MeOH to 100% MeOH in water with 0.1% FA), affording the product, tert-Butyl (2-(4-(4-((5-(m-tolyl)imidazo[1,2-a]pyrazin-8-yl)amino)phenyl)piperazin-1-yl)ethyl)carbamate (26 mg, 0.049 mmol, 48% yield), as a light yellow oil. LC/MS Method 4: MS (ESI) [M+H]⁺ 528.3, rt: 1.443 min.

N-[4-[4-(2-aminoethyl)piperazin-1-yl]phenyl]-5-(m-tolyl)imidazo[1,2-a]pyrazin-8-amine. To a solution of tert-butyl N-[2-[4-[4-[[5-(m-tolyl)imidazo[1,2-a]pyrazin-8-yl]amino]phenyl]piperazin-1-yl]ethyl]carbamate (26.0 mg, 0.050 mmol) in methanol (570 μL) was added HCl in dioxane (0.2 mL, 0.790 mmol), and the solution was stirred at room temperature for 24 h. LCMS showed full conversion to the desired product. The reaction was then concentrated and triturated with MeOH (3×) and MTBE (2×) to give the product N-[4-[4-(2-aminoethyl)piperazin-1-yl]phenyl]-5-(m-tolyl)imidazo[1,2-a]pyrazin-8-amine (21 mg, 0.045 mmol, 92% yield) as the hydrochloride salt, a yellow solid. LC/MS Method 1: MS (ESI) [M+H]⁺ 428.2, rt: 1.244 min.

2-((2-(2,6-Dioxopiperidin-3-yl)-1,3-dioxoisoindolin-5-yl)oxy)-N-(2-(4-(4-((5-(m-tolyl)imidazo[1,2-a]pyrazin-8-yl)amino)phenyl)piperazin-1-yl)ethyl)acetamide. To a solution of N-[4-[4-(2-aminoethyl)piperazin-1-yl]phenyl]-5-(m-tolyl)imidazo[1,2-a]pyrazin-8-amine (21.0 mg, 0.050 mmol), 2-[2-(2,6-dioxo-3-piperidyl)-1,3-dioxo-isoindolin-5-yl]oxyacetic acid (16.32 mg, 0.050 mmol) and DIPEA (0.09 mL, 0.490 mmol) in DMF (0.24 mL) was added PyAOP (31 mg, 0.060 mmol). The mixture was then stirred overnight at room temperature. LCMS showed full conversion to the desired product. The solution was then directly purified by reverse phase flash chromatography (5% to 100% MeCN in water with 0.1%), affording the product 2-((2-(2,6-Dioxopiperidin-3-yl)-1,3-dioxoisoindolin-5-yl)oxy)-N-(2-(4-(4-((5-(m-tolyl)imidazo[1,2-a]pyrazin-8-yl)amino)phenyl)piperazin-1-yl)ethyl)acetamide (28.6 mg, 0.038 mmol, 78% yield), as a yellow solid. LC/MS Method 3: MS (ESI) [M+H]⁺ 742.2, rt: 2.565 min. ¹H NMR (400 MHz, DMSO-d₆) δ ppm 1.96-2.04 (m, 1H), 2.39-2.42 (m, 3H), 2.42-2.47 (m, 2H), 2.52-2.63 (m, 6H), 2.81-2.94 (m, 1H), 3.02-3.10 (m, 4H), 4.75 (s, 2H), 5.11 (dd, J=12.6, 5.3 Hz, 1H), 6.91 (d, J=9.0 Hz, 2H), 7.33 (br d, J=6.6 Hz, 1H), 7.38-7.42 (m, 2H), 7.43-7.49 (m, 3H), 7.49-7.52 (m, 1H), 7.66 (s, 1H), 7.87 (dd, J=8.7, 3.8 Hz, 3H), 7.90-7.92 (m, 1H), 8.12-8.18 (m, 2H), 9.40 (s, 1H), 11.11 (s, 1H) (three protons not observed).

### Example S8. 2-((2-(2,6-Dioxopiperidin-3-yl)-1,3-dioxoisoindolin-5-yl)oxy)-N-(2-(4-(4-((5-(m-tolyl)imidazo[1,2-a]pyrazin-8-yl)amino)-1H-pyrazol-1-yl)piperidin-1-yl)ethyl)acetamide (8)

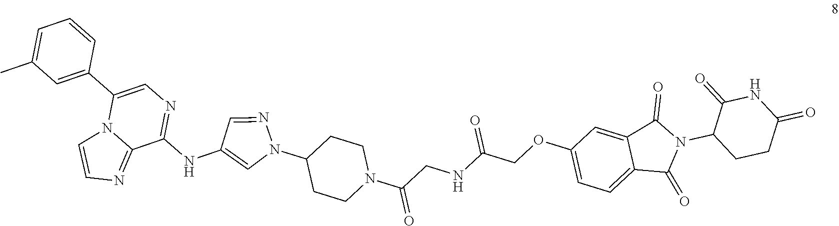 [SMILES: Cc1cccc(-c2cnc(Nc3cnn(C4CCN(C(=O)CNC(=O)COc5ccc6c(c5)C(=O)N(C5CCC(=O)NC5=O)C6=O)CC4)c3)c3nccn23)c1]

tert-Butyl N-[2-[4-[4-[[5-(m-tolyl)imidazo[1,2-a]pyrazin-8-yl]amino]pyrazol-1-yl]-1-piperidyl]ethyl]carbamate. To a solution of N-(1-(Piperidin-4-yl)-1H-pyrazol-4-yl)-5-(m-tolyl)imidazo[1,2-a]pyrazin-8-amine (300.0 mg, 0.67 mmol) and tert-butyl N-(2-oxoethyl)carbamate (168.92 mg, 1.01 mmol) in DCM (7 mL) was added DIPEA (0.47 mL, 2.69 mmol). The resulting mixture was stirred for 5 min at room temperature. NaBH(OAc)₃ (284.88 mg, 1.34 mmol) was then added and the reaction was stirred overnight at room temperature. After 18 h, LCMS showed around 70% conversion, an additional 0.5 equiv. of aldehyde and 1.0 equiv. of NaBH(OAc)₃ were added at room temperature. After 2 more hours, LCMS showed full conversion to the desired product. The mixture was then quenched with water and extracted with DCM, dried over sodium sulfate, filtered, and concentrated. The material was then purified by reverse phase column chromatography (5% to 100% MeOH in water with 0.1% FA), affording the product, tert-Butyl N-[2-[4-[4-[[5-(m-tolyl)imidazo[1,2-a]pyrazin-8-yl]amino]pyrazol-1-yl]-1-piperidyl]ethyl]carbamate (336 mg, 0.52 mmol, 77% yield), as a colorless oil. LC/MS Method 4: MS (ESI) [M+H]⁺ 517.4, rt: 1.526 min

N-(1-(1-(2-aminoethyl)piperidin-4-yl)-1H-pyrazol-4-yl)-5-(m-tolyl)imidazo[1,2-a]pyrazin-8-amine. To a solution of tert-butyl N-[2-[4-[4-[[5-(m-tolyl)imidazo[1,2-a]pyrazin-8-yl]amino]pyrazol-1-yl]-1-piperidyl]ethyl]carbamate (340.0 mg, 0.66 mmol) in methanol (6 mL) was added 4 μHCl in dioxane (3.0 mL, 6.57 mmol) at room temperature. The reaction mixture was stirred at room temperature, overnight. LCMS showed 80% conversion, an additional aliquot of 4 M HCl in dioxane (1 mL) was added and after 3 more hours, LCMS showed full conversion. The solvent was evaporated and co-evaporated with MTBE (3×), affording the desired product, N-(1-(1-(2-aminoethyl)piperidin-4-yl)-1H-pyrazol-4-yl)-5-(m-tolyl)imidazo[1,2-a]pyrazin-8-amine (280 mg, 0.57 mmol, 87% yield), as a light yellow solid. LC/MS Method 4: MS (ESI) [M+H]⁺ 417.2, rt: 1.458 min.

2-((2-(2,6-Dioxopiperidin-3-yl)-1,3-dioxoisoindolin-5-yl)oxy)-N-(2-(4-(4-((5-(m-tolyl)imidazo[1,2-a]pyrazin-8-yl)amino)-1H-pyrazol-1-yl)piperidin-1-yl)ethyl)acetamide. To a solution of N-(1-(1-(2-aminoethyl)piperidin-4-yl)-1H-pyrazol-4-yl)-5-(m-tolyl)imidazo[1,2-a]pyrazin-8-amine (200.0 mg, 0.41 mmol) in DMF (1.5 mL) at room temperature were added DIPEA (0.43 mL, 2.45 mmol) and 2-[2-(2,6-dioxo-3-piperidyl)-1,3-dioxo-isoindolin-5-yl]oxyacetic acid (136 mg, 0.410 mmol). The resulting solution was stirred at room temperature for 10 minutes. Then, PyAOP (213.0 mg, 0.41 mmol) was added in one portion. The reaction mixture was stirred at room temperature for 2 h. LCMS showed full conversion to the desired product. The mixture was directly purified by reverse phase column chromatography (5% MeCN to 100% MeCN in water with 0.1% FA), affording impure material. A second reverse phase purification was done (5 to 100% MeCN in water), affording the product, 2-((2-(2,6-dioxopiperidin-3-yl)-1,3-dioxoisoindolin-5-yl)oxy)-N-(2-(4-(4-((5-(m-tolyl)imidazo[1,2-a]pyrazin-8-yl)amino)-1H-pyrazol-1-yl)piperidin-1-yl)ethyl)acetamide (35 mg, 0.48 mmol, 11% yield), as a yellow solid. LC/MS Method 3: MS (ESI) [M+H]⁺ 731.3, rt: 2.381 min. ¹H NMR (400 MHz, DMSO-d₆) δ ppm 1.83-2.04 (m, 5H), 2.07-2.18 (m, 2H), 2.39-2.46 (m, 5H), 2.52-2.62 (m, 2H), 2.81-2.89 (m, 1H), 2.95 (br d, J=11.5 Hz, 2H), 3.26-3.30 (m, 2H), 4.05-4.20 (m, 1H), 4.75 (s, 2H), 5.12 (dd, J=12.7, 5.6 Hz, 1H), 7.28-7.35 (m, 1H), 7.38-7.52 (m, 6H), 7.64 (d, J=1.0 Hz, 1H), 7.80 (s, 1H), 7.86-7.90 (m, 2H), 8.14 (t, J=5.6 Hz, 1H), 8.22 (s, 1H), 9.88 (s, 1H), 11.12 (s, 1H).

### Example S9. 2-((2-(2,6-Dioxopiperidin-3-yl)-1,3-dioxoisoindolin-5-yl)oxy)-N-(2-(4-(3-((5-(m-tolyl)imidazo[1,2-a]pyrazin-8-yl)amino)phenyl)piperidin-1-yl)ethyl)acetamide (9)

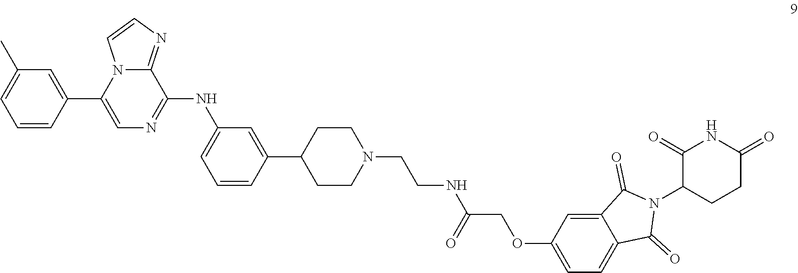 [SMILES: Cc1cccc(-c2cnc(Nc3cccc(C4CCN(CCNC(=O)COc5ccc6c(c5)C(=O)N(C5CCC(=O)NC5=O)C6=O)CC4)c3)c3nccn23)c1]

tert-Butyl 4-(3-((5-bromoimidazo[1,2-a]pyrazin-8-yl)amino)phenyl)piperidine-1-carboxylate. 5,8-Dibromoimidazo[1,2-a]pyrazine (165 mg, 0.6 mmol), tert-butyl 4-(3-aminophenyl)piperidine-1-carboxylate (198 mg, 0.72 mmol) and pivalic acid (0.67 mL, 5.96 mmol) were added to a vial and sealed with a septa cap. The mixture was stirred in an oil bath at 100° C. for 1 hour. HPLC showed complete conversion to the desired product. The resulting residue was cooled to room temperature and added to 100 mL of 10% aq. Na₂CO₃ solution and extracted with EtOAc (3×20 mL). The combined organics were dried over MgSO₄ and evaporated to afford the product, (303 mg, 0.574 mmol, 96% yield), as an orange oil, which was used directly in the next step. LC/MS Method 4: MS (ESI) [M]⁺ 472.2, rt: 2.092 min.

tert-Butyl 4-[3-[[5-(m-tolyl)imidazo[1,2-a]pyrazin-8-yl]amino]phenyl]piperidine-1-carboxylate. In a 30-mL microwave vial containing a stir bar, tert-butyl 4-(3-((5-bromoimidazo[1,2-a]pyrazin-8-yl)amino)phenyl)piperidine-1-carboxylate (303.0 mg, 0.64 mmol) was dissolved in 1,4-dioxane (3.78 mL) and water (0.49 mL). Then, m-tolylboronic acid (174 mg, 1.28 mmol) was added. The resulting solution was degassed with nitrogen for 10 minutes. After this, NaHCO₃ (0.04 mL, 1.92 mmol) and Xphos (24 mg, 0.05 mmol) were added, followed by Xphos Pd G3 (44 mg, 0.05 mmol). The system was degassed for 5 minutes and heated under microwave irradiation at 135° C. for 45 min. The resulting black suspension was filtered in celite and the resulting solution was evaporated; the residue was purified by reverse phase flash chromatography (5% to 100% MeOH in water with 0.1% FA), affording the product, (217 mg, 0.448 mmol, 70% yield), as an orange semi-solid. LC/MS Method 4: MS (ESI) [M+H]⁺ 484.2, rt: 2.205 min.

N-(3-(piperidin-4-yl)phenyl)-5-(m-tolyl)imidazo[1,2-a]pyrazin-8-amine. tert-Butyl 4-(3-((5-(m-tolyl)imidazo[1,2-a]pyrazin-8-yl)amino)phenyl)piperidine-1-carboxylate (217.0 mg, 0.45 mmol) was dissolved in methanol (4.49 mL). A 4.0 M solution of HCl in dioxane (2.24 mL, 8.97 mmol) was added and the resulting solution was stirred at room temperature. The reaction was monitored by HPLC. After 3 h, the reaction was complete. The solvent was evaporated in vacuo and the residue was chased off with MeCN (2×5 mL) to form a colorless solid. The residue was purified by reverse phase flash chromatography (5 to 100% MeOH in 0.1% FA), affording the product, N-(3-(piperidin-4-yl)phenyl)-5-(m-tolyl)imidazo[1,2-a]pyrazin-8-amine (152 mg, 0.354 mmol, 79%), a white solid. LC/MS Method 4: MS (ESI) [M+H]⁺ 384.2, rt: 1.425 min.

tert-Butyl (2-(4-(3-((5-(m-tolyl)imidazo[1,2-a]pyrazin-8-yl)amino)phenyl)piperidin-1-yl)ethyl)carbamate. N-(3-(piperidin-4-yl)phenyl)-5-(m-tolyl)imidazo[1,2-a]pyrazin-8-amine (152.0 mg, 0.35 mmol) and tert-butyl N-(2-oxoethyl)carbamate (84.5 mg, 0.53 mmol) were dissolved in DCE (3.53 mL). DIPEA (0.07 mL, 0.39 mmol) and NaBH(OAc)₃ (90.0 mg, 0.42 mmol) were added to the reaction vessel. The resulting suspension was stirred overnight at room temperature. LCMS analysis indicated the reaction went to 70% conversion, therefore an additional 20 mg of the aldehyde and 20 mg of NaBH(OAc)₃ were added. After 3 hours, the solvent was removed in vacuo and the residue was purified by reverse phase flash chromatography (5% to 100% MeOH in water with 0.1% FA), affording the product, tert-butyl (2-(4-(3-((5-(m-tolyl)imidazo[1,2-a]pyrazin-8-yl)amino)phenyl)piperidin-1-yl)ethyl)carbamate (176 mg, 0.297 mmol, 84%), as a colorless semi-solid. LC/MS Method 4: MS (ESI) [M+2H]²⁺ 264.4, rt: 1.531 min.

N-(3-(1-(2-aminoethyl)piperidin-4-yl)phenyl)-5-(m-tolyl)imidazo[1,2-a]pyrazin-8-amine. tert-butyl (2-(4-(3-((5-(m-tolyl)imidazo[1,2-a]pyrazin-8-yl)amino)phenyl)piperidin-1-yl)ethyl)carbamate (175.0 mg, 0.33 mmol) was dissolved in methanol (3.3 mL) then 4.0 M HCl in dioxane (1.66 mL, 6.65 mmol) was added and the resulting solution was stirred at room temperature. After 3 h, LCMS analysis revealed that the reaction went to completion. The solvent was evaporated in vacuo and the residue was chased off with MeOH (2×5 mL), affording the product, N-(3-(1-(2-aminoethyl)piperidin-4-yl)phenyl)-5-(m-tolyl)imidazo[1,2-a]pyrazin-8-amine (166 mg, 0.332 mmol, 100% yield), as a brown solid. LC/MS Method 4: MS (ESI) [M+H]⁺ 427.3, rt: 1.330 min.

2-((2-(2,6-Dioxopiperidin-3-yl)-1,3-dioxoisoindolin-5-yl)oxy)-N-(2-(4-(3-((5-(m-tolyl)imidazo[1,2-a]pyrazin-8-yl)amino)phenyl)piperidin-1-yl)ethyl)acetamide. N-(3-(1-(2-aminoethyl)piperidin-4-yl)phenyl)-5-(m-tolyl)imidazo[1,2-a]pyrazin-8-amine (166.0 mg, 0.33 mmol) and 2-[2-(2,6-dioxo-3-piperidyl)-1,3-dioxo-isoindolin-5-yl]oxyacetic acid (115.95 mg, 0.3500 mmol) were dissolved in DMF (2.2156 mL), followed by DIPEA (0.29 mL, 1.66 mmol). The resulting solution was stirred for 5 min and then cooled using an ice bath; then, solid PyAOP (191 mg, 0.37 mmol) was added. The reaction was stirred for 15 minutes in the ice bath and then it was stirred for 1 hour at room temperature. The reaction was then directly purified by reverse phase column chromatography (5% MeCN to 40% MeCN in water with 0.1% FA). The collected material was impure and a second reverse phase purification (5 to 100% ACN in water) was carried out. Affording the desired product, 2-((2-(2,6-Dioxopiperidin-3-yl)-1,3-dioxoisoindolin-5-yl)oxy)-N-(2-(4-(3-((5-(m-tolyl)imidazo[1,2-a]pyrazin-8-yl)amino)phenyl)piperidin-1-yl)ethyl)acetamide (27.3 mg, 0.036 mmol, 11% yield), as a light yellow solid. LC/MS Method 3: MS (ESI) [M+H]⁺ 741.2, rt: 2.619 min. ¹H NMR (400 MHz, DMSO-d₆) δ ppm 1.64 (qd, J=12.2, 2.9 Hz, 2H), 1.72-1.80 (m, 2H), 1.96-2.02 (m, 1H), 2.07 (td, J=11.5, 1.5 Hz, 2H), 2.41 (s, 3H), 2.42-2.48 (m, 3H), 2.54 (s, 2H), 2.81-2.96 (m, 1H), 2.97 (br d, J=11.2 Hz, 2H), 4.75 (s, 2H), 5.11 (dd, J=13.0, 5.4, 1H), 6.89 (d, J=7.8 Hz, 1H), 7.25 (t, J=7.8 Hz, 1H), 7.34 (d, J=7.1 Hz, 1H), 7.40 (dd, J=8.3, 2.4 Hz, 1H), 7.44-7.48 (m, 3H), 7.51 (br d, J=13.7 Hz, 2H), 7.69 (d, J=1.0 Hz, 1H), 7.87 (dd, J=8.3 Hz, 1H), 7.91-7.94 (m, 1H), 7.94 (d, J=1.2 Hz, 2H), 8.10-8.16 (m, 1H), 9.47 (s, 1H), 11.11 (s, 1H), (three protons not observed).

### Example S10. 2-((2-(2,6-Dioxopiperidin-3-yl)-1,3-dioxoisoindolin-5-yl)oxy)-N-(2-(4-(4-((5-(m-tolyl)imidazo[1,2-a]pyrazin-8-yl)amino)phenyl)piperidin-1-yl)ethyl)acetamide (10)

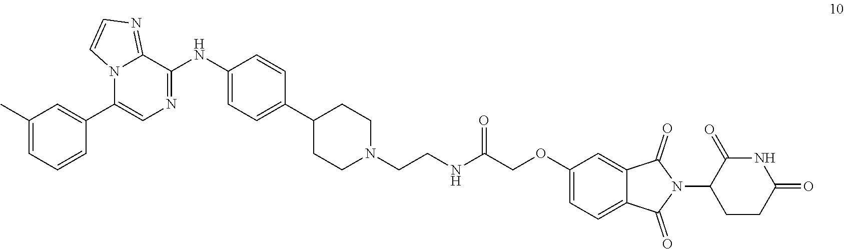 [SMILES: Cc1cccc(-c2cnc(Nc3ccc(C4CCN(CCNC(=O)COc5ccc6c(c5)C(=O)N(C5CCC(=O)NC5=O)C6=O)CC4)cc3)c3nccn23)c1]

tert-Butyl 4-[4-[(5-bromoimidazo[1,2-a]pyrazin-8-yl)amino]phenyl]piperidine-1-carboxylate. 5,8-Dibromoimidazo[1,2-a]pyrazine (250 mg, 0. mmol), tert-butyl 4-(4-aminophenyl)piperidine-1-carboxylate (299 mg, 1.08 mmol) and pivalic acid (1.02 mL, 9.03 mmol) were added to a vial and sealed with a septa. The resulting mixture was stirred in an oil bath at 100° C. for 1 hour. HPLC analysis showed complete conversion to the desired product. The resulting residue was cooled to room temperature and then ca. 20 mL of heptane was added, and the suspension was stirred for 10 minutes. The suspension was filtered using a Buchner funnel and the solid was washed twice with heptane. The resulting solid was dried for 3 hours in the vacuum pump to afford the desired product, tert-butyl 4-[4-[(5-bromoimidazo[1,2-a]pyrazin-8-yl)amino]phenyl]piperidine-1-carboxylate (415 mg, 0.8 mmol, 89% yield), as a light-yellow solid. LC/MS Method 4: MS (ESI) [M]⁺ 472.1, rt: 2.088 min.

tert-Butyl 4-(4-((5-(m-tolyl)imidazo[1,2-a]pyrazin-8-yl)amino)phenyl)piperidine-1-carboxylate. In a 30-mL microwave vial containing a stir bar, tert-butyl 4-[4-[(5-bromoimidazo[1,2-a]pyrazin-8-yl)amino]phenyl]piperidine-1-carboxylate (250 mg, 0.53 mmol) was dissolved in 1,4-dioxane (2.8 mL). Then m-tolylboronic acid (144 mg, 1.06 mmol) was added. The resulting solution was degassed with nitrogen for 10 minutes. After this, 2.0 M aq. sodium bicarbonate (133 mg, 1.59 mmol) and Xphos Pd G3 (36 mg, 0.04 mmol) were added, followed by Xphos (20. mg, 0.04 mmol). The system was degassed for 5 minutes and was then heated at 135° C. under microwave irradiation for 45 min. After this, the resulting black suspension was filtered over celite and the resulting solution was concentrated. The residue was purified by reverse phase column chromatography (5 to 100% MeOH in water with 0.1% FA), affording the product, tert-Butyl 4-(4-((5-(m-tolyl)imidazo[1,2-a]pyrazin-8-yl)amino)phenyl)piperidine-1-carboxylate (179 mg, 0.37 mmol, 70% yield), as a white solid. LC/MS Method 4: MS (ESI) [M+H]⁺ 484.4, rt: 2.191 min.

N-(4-(piperidin-4-yl)phenyl)-5-(m-tolyl)imidazo[1,2-a]pyrazin-8-amine. tert-Butyl 4-(4-((5-(m-tolyl)imidazo[1,2-a]pyrazin-8-yl)amino)phenyl)piperidine-1-carboxylate (179 mg, 0.37 mmol) was dissolved in methanol (3.7 mL) and 4.0 M HCl in dioxane (1.85 mL, 7.4 mmol) was added, the resulting solution was stirred at room temperature. After 3 h, HPLC analysis revealed the reaction was complete. The solvent was evaporated in vacuo and the residue was chased off with MeCN (2×5 mL), affording the product, N-(4-(piperidin-4-yl)phenyl)-5-(m-tolyl)imidazo[1,2-a]pyrazin-8-amine (quantitative), as a yellow solid. LC/MS Method 4: MS (ESI) [M+H]⁺ 384.5, rt: 1.391 min.

tert-Butyl (2-(4-(4-((5-(m-tolyl)imidazo[1,2-a]pyrazin-8-yl)amino)phenyl)piperidin-1-yl)ethyl)carbamate. N-(4-(Piperidin-4-yl)phenyl)-5-(m-tolyl)imidazo[1,2-a]pyrazin-8-amine (209 mg, 0.46 mmol) was suspended in DCE (5.0 mL) and then DIPEA (0.17 mL, 0.96 mmol) was added followed by tert-butyl N-(2-oxoethyl)carbamate (109 mg, 0.69 mmol). After 10 minutes, NaBH(OAc)₃ (116 mg, 0.55 mmol) was added and the reaction was stirred at room temperature. After 3 hours, 20% conversion was observed; the reaction was then heated at 40° C. for 1 hour and stirred overnight at room temperature. Silica gel was added to the resulting solution and the solvent was removed in vacuo. The obtained residue was purified by normal phase column chromatography (0 to 10% MeOH in DCM, affording the product, tert-butyl (2-(4-(4-((5-(m-tolyl)imidazo[1,2-a]pyrazin-8-yl)amino)phenyl)piperidin-1-yl)ethyl)carbamate (76 mg 0.13 mmol, 29% yield), as a clear oil. LC/MS Method 4: MS (ESI) [M+H]⁺ 527.4, rt: 1.508 min.

N-(4-(1-(2-Aminoethyl)piperidin-4-yl)phenyl)-5-(m-tolyl)imidazo[1,2-a]pyrazin-8-amine. tert-Butyl (2-(4-(4-((5-(m-tolyl)imidazo[1,2-a]pyrazin-8-yl)amino)phenyl)piperidin-1-yl)ethyl)carbamate (75. mg, 0.1400 mmol) was dissolved in methanol (1.9 mL), then 4.0 M HCl in dioxane (0.71 mL, 2.85 mmol) was added and the resulting solution was stirred at room temperature. After 3 h, HPLC analysis showed the reaction was complete. The solvent was evaporated in vacuo and the residue was chased off with MeCN (2×5 mL), affording the product, N-(4-(1-(2-aminoethyl)piperidin-4-yl)phenyl)-5-(m-tolyl)imidazo[1,2-a]pyrazin-8-amine (76 mg, 0.144, 100% yield), as a light-yellow solid. LC/MS Method 4: MS (ESI) [M+2H]²⁺ 214.4, rt: 1.337 min.

2-((2-(2,6-dioxopiperidin-3-yl)-1,3-dioxoisoindolin-5-yl)oxy)-N-(2-(4-(4-((5-(m-tolyl)imidazo[1,2-a]pyrazin-8-yl)amino)phenyl)piperidin-1-yl)ethyl)acetamide. N-(4-(1-(2-aminoethyl)piperidin-4-yl)phenyl)-5-(m-tolyl)imidazo[1,2-a]pyrazin-8-amine (74.0 mg, 0.15 mmol) and 2-[2-(2,6-dioxo-3-piperidyl)-1,3-dioxo-isoindolin-5-yl]oxyacetic acid (51.69 mg, 0.16 mmol) were dissolved in DMF (1.0 mL) then DIPEA (0.13 mL, 0.74 mmol) was added. The resulting solution was stirred for 5 minutes and then cooled using an ice bath. PyAOP (85 mg, 0.16 mmol) was then added. The reaction was stirred for 15 minutes in the ice bath and then it was stirred for 1 hour at room temperature. The product was then purified by reverse phase flash chromatography (5% to 100% MeCN in water with 0.1% FA), affording the product, 2-((2-(2,6-dioxopiperidin-3-yl)-1,3-dioxoisoindolin-5-yl)oxy)-N-(2-(4-(4-((5-(m-tolyl)imidazo[1,2-a]pyrazin-8-yl)amino)phenyl)piperidin-1-yl)ethyl)acetamide (2.7 mg, 0.0036 mmol, 2.4% yield), as a light-yellow solid. LC/MS Method 3: MS (ESI) [M+H]⁺ 741.2, rt: 2.598 min. ¹H NMR (400 MHz, DMSO-d₆) δ ppm 1.56-1.67 (m, 2H), 1.68-1.76 (m, 2H), 1.94-2.08 (m, 3H), 2.39-2.44 (m, 6H), 2.53-2.62 (m, 2H), 2.81-2.91 (m, 1H), 2.92-3.00 (m, 2H), 4.75 (s, 2H), 5.12 (dd, J=13.0, 5.4 Hz, 1H), 7.18 (d, J=8.8 Hz, 2H), 7.32-7.38 (m, 1H), 7.39-7.42 (m, 1H), 7.42 (s, 1H), 7.44-7.49 (m, 3H), 7.52 (s, 1H), 7.68 (d, J=1.2 Hz, 1H), 7.88 (d, J=8.3 Hz, 1H), 7.91-7.95 (m, 2H), 7.96 (s, 1H), 8.13 (t, J=5.7 Hz, 1H), 9.53 (s, 1H), 11.11 (s, 1H), (two protons not observed).

### Example S11. 2-((2-(2,6-Dioxopiperidin-3-yl)-1,3-dioxoisoindolin-5-yl)oxy)-N-(2-(4-(3-((5-(m-tolyl)imidazo[1,2-a]pyrazin-8-yl)amino)phenyl)piperazin-1-yl)ethyl)acetamide (11)

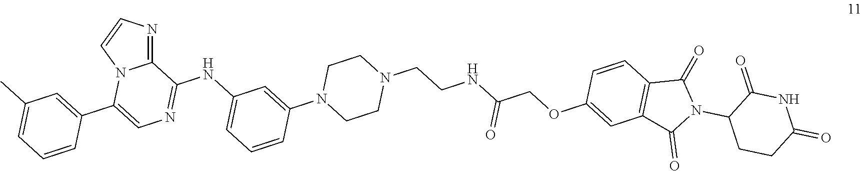 [SMILES: Cc1cccc(-c2cnc(Nc3cccc(N4CCN(CCNC(=O)COc5ccc6c(c5)C(=O)N(C5CCC(=O)NC5=O)C6=O)CC4)c3)c3nccn23)c1]

tert-Butyl 4-(3-((5-bromoimidazo[1,2-a]pyrazin-8-yl)amino)phenyl)piperazine-1-carboxylate. To a solution of tert-butyl 4-(3-aminophenyl)piperazine-1-carboxylate (1 g, 3.61 mmol) and 5,8-dibromoimidazo[1,2-a]pyrazine (0.9 g, 3.24 mmol) in NMP (6 mL) at room temperature was added DIPEA (1.3 mL, 7.45 mmol). The reaction flask was placed in an oil bath pre-equilibrated to 100° C. and was stirred for 48 h. The reaction was cooled to room temperature and added to 100 mL of sat. aq. NaHCO₃ solution. The resulting mixture was extracted with EtOAc (3×20 mL). The combined organic layers were washed with brine (1×50 mL), dried over Na₂SO₄, and evaporated to form 5.93 g of a black oil. This was purified using reverse phase column chromatography (5% to 100% MeOH in water with 0.1% FA), affording the product, tert-butyl 4-(3-((5-bromoimidazo[1,2-a]pyrazin-8-yl)amino)phenyl)piperazine-1-carboxylate (750 mg, 1.47 mmol, 41% yield) as a brown solid.

tert-Butyl 4-(3-((5-(m-tolyl)imidazo[1,2-a]pyrazin-8-yl)amino)phenyl)piperazine-1-carboxylate. A dry seal tube was charged with tert-butyl 4-(3-((5-bromoimidazo[1,2-a]pyrazin-8-yl)amino)phenyl)piperazine-1-carboxylate (500 mg, 0.96 mmol) and m-tolylboronic acid (158 mg, 1.17 mmol) at room temperature. Then 1,4-dioxane (10 mL) and sat. aq. NaHCO₃ (7.82 mL, 7.82 mmol) were added and the mixture was sparged with nitrogen for 10 minutes. Then Pd(PPh₃)₄ (58 mg, 0.05 mmol) was added in one portion and the sparging with nitrogen was continued for 5 minutes. The tube was sealed and placed in an oil bath pre-equilibrated to 90° C. The mixture was heated overnight at that temperature. LCMS analysis revealed complete conversion to the desired product. The reaction mixture was diluted with water (50 mL) and extracted with EtOAc (3×150 mL). The combined organic layers were washed with brine, dried over MgSO₄, filtered, and concentrated to give a crude residue, which was purified by normal phase chromatography (70% EtOAc in heptane), which afforded the desired product, tert-Butyl 4-(3-((5-(m-tolyl)imidazo[1,2-a]pyrazin-8-yl)amino)phenyl)piperazine-1-carboxylate (quantitative yield, 91% pure). LC/MS Method 4: MS (ESI) [M+H]⁺ 485.4, rt: 2.082 min.

N-(3-(Piperazin-1-yl)phenyl)-5-(m-tolyl)imidazo[1,2-a]pyrazin-8-amine. tert-Butyl 4-(3-((5-(m-tolyl)imidazo[1,2-a]pyrazin-8-yl)amino)phenyl)piperazine-1-carboxylate (600 mg, 1.24 mmol) was dissolved in methanol (8 mL). A solution of 4 M HCl in dioxane (3.1 mL, 12.4 mmol) was then added at room temperature. The reaction mixture was stirred overnight at room temperature. LCMS analysis revealed complete conversion to the desired product. Overnight, a precipitate formed which was filtered out and washed with MeOH, which afforded the desired product, N-(3-(Piperazin-1-yl)phenyl)-5-(m-tolyl)imidazo[1,2-a]pyrazin-8-amine (285 mg, 0.73 mmol, 59% yield), as yellow solid. LC/MS Method 3: MS (ESI) [M+H]⁺ 385.2, rt: 2.558 min.

tert-Butyl (2-(4-(3-((5-(m-tolyl)imidazo[1,2-a]pyrazin-8-yl)amino)phenyl)piperazin-1-yl)ethyl)carbamate. tert-Butyl N-(2-oxoethyl)carbamate (41.6 mg, 0.26 mmol) was mixed with N-(3-(piperazin-1-yl)phenyl)-5-(m-tolyl)imidazo[1,2-a]pyrazin-8-amine (75 mg, 0.17 mmol) in DCE (1.74 mL) and DIPEA (0.03 mL, 0.19 mmol) then NaBH(OAc)₃ (44 mg, 0.21 mmol) was added, and the suspension was stirred overnight at room temperature. HPLC analysis revealed ˜95% conversion to the desired product. The solvent was removed in vacuo and the residue was purified by normal phase column chromatography (0 to 10% MeOH in DCM), affording the product, tert-butyl (2-(4-(3-((5-(m-tolyl)imidazo[1,2-a]pyrazin-8-yl)amino)phenyl)piperazin-1-yl)ethyl)carbamate (76 mg, 0.144 mmol, 82% yield), as a colorless semi-solid. LC/MS Method 4: MS (ESI) [M+2H]²⁺ 264.8, rt: 1.519 min.

N-(3-(4-(2-Aminoethyl)piperazin-1-yl)phenyl)-5-(m-tolyl)imidazo[1,2-a]pyrazin-8-amine. tert-Butyl (2-(4-(3-((5-(m-tolyl)imidazo[1,2-a]pyrazin-8-yl)amino)phenyl)piperazin-1-yl)ethyl)carbamate (76 mg, 0.14 mmol) was dissolved in methanol (1.4 mL) then 4.0 M HCl in dioxane (0.71 mL, 2.84 mmol) was added and the resulting solution was stirred at room temperature. After 3 h, HPLC analysis revealed that the reaction was complete. The solvent was evaporated in vacuo and the residue was chased off with MeCN (2×5 mL), affording the product, N-(3-(4-(2-aminoethyl)piperazin-1-yl)phenyl)-5-(m-tolyl)imidazo[1,2-a]pyrazin-8-amine (quantitative), as a colorless semi-solid. LC/MS Method 4: MS (ESI) [M+H]⁺ 428.2, rt: 1.392 min.

2-((2-(2,6-Dioxopiperidin-3-yl)-1,3-dioxoisoindolin-5-yl)oxy)-N-(2-(4-(3-((5-(m-tolyl)imidazo[1,2-a]pyrazin-8-yl)amino)phenyl)piperazin-1-yl)ethyl)acetamide. N-(3-(4-(2-aminoethyl)piperazin-1-yl)phenyl)-5-(m-tolyl)imidazo[1,2-a]pyrazin-8-amine (76 mg, 0.15 mmol) and the 2-[2-(2,6-dioxo-3-piperidyl)-1,3-dioxo-isoindolin-5-yl]oxyacetic acid (55.5 mg, 0.17 mmol) were dissolved in DMF (0.76 mL) then DIPEA (0.11 mL, 0.6100 mmol) was added. The resulting solution was stirred for 5 minutes and HATU (69 mg, 0.18 mmol) was added. The reaction was stirred for 1 hour at room temperature. The residue was then purified by reverse-phase column chromatography (5% to 100% MeCN in water with 0.1% FA). The resulting material contained impurities and was repurified by reverse-phase column chromatography (5% to 100% MeCN in water), affording the product, 2-((2-(2,6-dioxopiperidin-3-yl)-1,3-dioxoisoindolin-5-yl)oxy)-N-(2-(4-(3-((5-(m-tolyl)imidazo[1,2-a]pyrazin-8-yl)amino)phenyl)piperazin-1-yl)ethyl)acetamide (13.3 mg, 0.0179 mmol, 12% yield), as a yellow solid. LC/MS Method 3: MS (ESI) [M+H]⁺ 742.2, rt: 2.605 min. ¹H NMR (400 MHz, DMSO-d₆) δ ppm 1.94-2.06 (m, 1H), 2.41 (s, 3H), 2.43-2.48 (m, 2H), 2.53-2.62 (m, 4H), 2.81-2.95 (m, 1H), 3.11 (br s, 4H), 4.75 (s, 2H), 5.11 (dd, J=12.7, 5.4 Hz, 1H), 6.60 (d, J=7.8 Hz, 1H), 7.15 (t, J=8.1 Hz, 1H), 7.34 (d, J=7.3 Hz, 1H), 7.40 (dd, J=8.3, 2.4 Hz, 1H), 7.43-7.46 (m, 2H), 7.46-7.51 (m, 2H), 7.52 (s, 1H), 7.60-7.67 (m, 2H), 7.69 (d, J=1.0 Hz, 1H), 7.87 (d, J=8.3 Hz, 1H), 7.94 (d, J=1.2 Hz, 1H), 8.14 (t, J=4.9 Hz, 1H), 9.33 (s, 1H), 11.13 (s, 1H), (four protons obscured by DMSO peak).

### Example S12. 3-(1-Oxo-5-(9-(4-(4-((5-(m-tolyl)imidazo[1,2-a]pyrazin-8-yl)amino)-1H-pyrazol-1-yl)piperidin-1-yl)-3-azaspiro[5.5]undecane-3-carbonyl)isoindolin-2-yl)piperidine-2,6-dione (12)

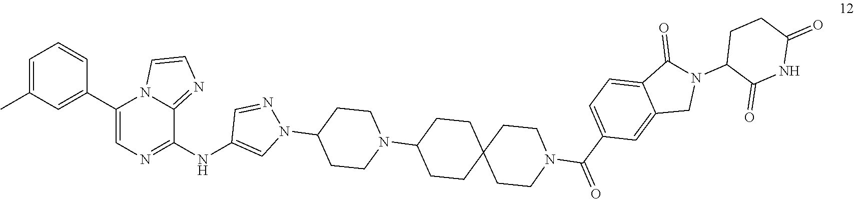 [SMILES: Cc1cccc(-c2cnc(Nc3cnn(C4CCN(C5CCC6(CC5)CCN(C(=O)c5ccc7c(c5)CN(C5CCC(=O)NC5=O)C7=O)CC6)CC4)c3)c3nccn23)c1]

tert-Butyl 9-(4-(4-((5-(m-tolyl)imidazo[1,2-a]pyrazin-8-yl)amino)-1H-pyrazol-1-yl)piperidin-1-yl)-3-azaspiro[5.5]undecane-3-carboxylate. To a solution of N-(1-(piperidin-4-yl)-1H-pyrazol-4-yl)-5-(m-tolyl)imidazo[1,2-a]pyrazin-8-amine (370 mg, 0.99 mmol) and tert-butyl 9-oxo-3-azaspiro[5.5]undecane-3-carboxylate (344 mg, 1.29 mmol) in DCE (10 mL) was added NaBH(OAc)₃ (315 mg, 1.49 mmol). The reaction mixture was stirred at room temperature. After 1.5, LCMS showed ˜30% of starting material remained. DIPEA (0.35 mL, 2 mmol) was added, and the reaction mixture was stirred overnight at room temperature. LCMS showed ˜16% of starting material remained. Ketone (0.3 equiv.) and NaBH(OAc)₃ (0.3 equiv.) were added, and the reaction was stirred for another 2 h. The reaction was quenched with water and diluted with EtOAc. The phases were separated and extracted with EtOAc. The organic layers were washed with brine, dried over sodium sulfate, filtered, and concentrated. The crude material was purified by reverse phase column chromatography (5% to 100% MeOH in water with 0.1% FA), affording the product, tert-butyl 9-(4-(4-((5-(m-tolyl)imidazo[1,2-a]pyrazin-8-yl)amino)-1H-pyrazol-1-yl)piperidin-1-yl)-3-azaspiro[5.5]undecane-3-carboxylate (420 mg, 0.62 mmol, 63% yield, formic acid salt) as a yellow semi-solid. LC/MS Method 4: MS (ESI) [M+H]⁺ 625.4, rt: 1.529 min.

N-(1-(1-(3-azaspiro[5.5]undecan-9-yl)piperidin-4-yl)-1H-pyrazol-4-yl)-5-(m-tolyl)imidazo[1,2-a]pyrazin-8-amine. To a solution of tert-butyl 9-[4-[4-[[5-(m-tolyl)imidazo[1,2-a]pyrazin-8-yl]amino]pyrazol-1-yl]-1-piperidyl]-3-azaspiro[5.5]undecane-3-carboxylate (418.0 mg, 0.62 mmol) in methanol (6 mL) was added 4 M HCl in dioxane (1.6 mL, 6.4 mmol). The resulting solution was stirred overnight at room temperature. HPLC analysis showed complete conversion. The volatiles were removed in vacuo and the crude was purified by reverse phase column chromatography (5% to 100% MeOH in water with pH 10 buffer), affording the product, N-(1-(1-(3-azaspiro[5.5]undecan-9-yl)piperidin-4-yl)-1H-pyrazol-4-yl)-5-(m-tolyl)imidazo[1,2-a]pyrazin-8-amine (300 mg, 0.57 mmol, 92% yield), as a light yellow solid. LC/MS Method 4: MS (ESI) [M+H]⁺ 525.3, rt: 1.239 min.

3-(1-Oxo-5-(9-(4-(4-((5-(m-tolyl)imidazo[1,2-a]pyrazin-8-yl)amino)-1H-pyrazol-1-yl)piperidin-1-yl)-3-azaspiro[5.5]undecane-3-carbonyl)isoindolin-2-yl)piperidine-2,6-dione. To a solution of 2-(2,6-dioxopiperidin-3-yl)-1-oxoisoindoline-5-carboxylic acid (103 mg, 0.36 mmol) in DMF (3 mL) was added HATU (159 mg, 0.42 mmol) followed by a solution of N-(1-(1-(3-azaspiro[5.5]undecan-9-yl)piperidin-4-yl)-1H-pyrazol-4-yl)-5-(m-tolyl)imidazo[1,2-a]pyrazin-8-amine (208 mg, 0.35 mmol) and DIPEA (0.61 mL, 3.48 mmol) in DMF (2 mL). The mixture was stirred at room temperature for 1 h. LCMS analysis showed complete conversion. The mixture was purified by reverse phase column chromatography (5% to 100% MeCN in Water with 0.1% FA), affording the product 3-(1-oxo-5-(9-(4-(4-((5-(m-tolyl)imidazo[1,2-a]pyrazin-8-yl)amino)-1H-pyrazol-1-yl)piperidin-1-yl)-3-azaspiro[5.5]undecane-3-carbonyl)isoindolin-2-yl)piperidine-2,6-dione (100 mg, 0.126 mmol, 36% yield), as a light yellow solid. LC/MS Method 3: MS (ESI) [M+H]⁺ 795.3, rt: 2.391 min. ¹H NMR (400 MHz, DMSO-d₆) δ ppm 1.05-1.20 (m, 2H), 1.22-1.29 (m, 1H), 1.33-1.50 (m, 4H), 1.52-1.65 (m, 3H), 1.75-1.82 (m, 2H), 1.86-1.95 (m, 2H), 1.98-2.06 (m, 3H), 2.32-2.43 (m, 7H), 2.61 (br d, J=17.6 Hz, 1H), 2.86-2.94 (m, 1H), 2.95-3.03 (m, 2H), 3.24-3.27 (m, 2H), 3.58-3.65 (m, 2H), 4.06-4.16 (m, 1H), 4.38 (d, J=17.7 Hz, 1H), 4.50 (d, J=17.7 Hz, 1H), 5.13 (br dd, J=13.4, 5.1 Hz, 1H), 7.32 (br d, J=6.4 Hz, 1H), 7.40-7.51 (m, 5H), 7.60-7.65 (m, 2H), 7.76-7.81 (m, 2H), 7.89 (s, 1H), 8.16 (s, 1H), 8.23 (br s, 1H), 9.88 (s, 1H), 11.00 (s, 1H), (one proton not observed in spectrum).

### Biological Examples

### Example B1. IRAK3-ePL Overexpressing Degradation Assay

Stable cell lines were generated by the following protocol. 3×10⁵ Lenti-X 293T cells (Clonetech) were plated in 0.8 mL of media in a 12-well plate and incubated overnight at 37° C./5% CO₂. Packaging plasmid (0.4 μg, pMD), envelope plasmid (0.4 μg, pSP), and lentiviral transfer IRAK3-ePL plasmid (0.8 μg, IRAK3 sequence NM_007199.3) were mixed in 0.1 mL of Opti-MEM and incubated for 5 min. Simultaneously, 2.4 μL of Lipofectamine 2000 (Invitrogen) was added to 0.1 mL of Opti-MEM (Gibco) and incubated for 5 min. The plasmid DNA and lipofectamine were combined and the mixture was allowed to incubate for 20 min.DNA:Lipofectamine Opti-MEM mixtures were then added to previously plated cells dropwise and the cells were incubated for ˜16 h at 37° C./5% CO₂. Following incubation, the media was removed and 1.2 mL of fresh media was added per well. Lenti-X 293T cells were incubated for ˜30 h at 37° C./5% CO₂. 0.5×10⁶ 293T^{CRBN OE/GSPT1 G575N KI} cells were plated in 0.5 mL of media/well of a 12-well plate and incubated for ˜16 h at 37° C./5% CO₂. Following incubation, media was removed from the Lenti-X 293T wells and passed through a 0.45 μM filter. Part of the viral supernatant was used to transduce cells and the rest was stored at −80° C. Viruses were then added individually (0.5 mL virus) to each well of the 293T^{CRBN OE/GSPT1 G575N KI} cells, followed by addition of polybrene (10 mg/mL Millipore) to each well at a final concentration of 5.0 μg/mL. Cells were incubated for ˜24 h at 37° C./5% CO₂. After aspirating media off plates, cells were washed with DPBS, trypsinized and plated in a 10 cm dish in 15 mL of media and 1 μg/mL puromycin. Following incubation of cells for ˜72 h at 37° C./5% CO₂, media was aspirated off plates, and cells were washed with DPBS and trypsinized. The cells were plated in a 15 cm dish in 40 mL of media with 1.0 μg/mL Puromycin (Gibco) and incubated for ˜72 h at 37° C./5% CO₂. Following incubation, media was removed and cells were washed with DPBS and trypsinized. The majority of cells were resuspended in Invitrogen freezing media and stored away (˜6-8×10⁶ cells/vial).

IRAK3-ePL cellular dose response curve degradation assays were performed by the following protocol. Compounds to be tested were dispensed into a white 384-well tissue-culture treated plate using an acoustic liquid handler. Dilutions were prepared based on a 25 μL assay volume in duplicate 10 point 3-fold serial dilutions starting with a 10 μM dose. Negative control wells were included, which only contain 0.2% DMSO to calculate 100% signal. Positive control wells containing 30 μM Ataluren (luciferase inhibitor) were included to calculate the background signal level. All wells were backfilled to a final DMSO concentration of 0.2% to ensure DMSO uniformity across wells. IRAK3-ePL expressing cells (IRAK3-ePL Lenti-X 293T^{CRBN/GSPT1 G575N}) were washed, trypsinized, counted, and resuspended in fresh DMEM (Gibco) to give a cell concentration of 200,000 cells/mL. 25 μL of cells (5,000 cells/well) were dispensed into the wells of the 384-well plate prespotted with compounds in the previous step and incubated overnight at 37° C./5% CO₂. Following incubation, the 384-well plate was taken out of the incubator and left at room temperature for 30 min. InCELL hunter reagent was prepared according to manufacturer's instructions (EA reagent, lysis buffer, and substrate reagent in a 1:1:4 ratio, Cat #96-0002, DiscoverX), which was added to the 384-well plate 25 μL per well. Following the incubation of the plate for 1 h at room temperature, the luminescence signal was read using a ViewLux plate reader. Data was processed and analyzed in ActivityBase software. In short, the average luminescence values of the positive control wells were subtracted from the rest of the wells for background correction, and all luminescence values were normalized to the DMSO control wells. The average value of the DMSO control wells was set to equal 100% of the relative IRAK3-ePL protein levels. Normalized luminescence values were plotted on a graph as a function of compound concentration. Compound concentration was plotted on the x-axis and the corresponding relative IRAK3-ePL protein levels on the y-axis. The EC₅₀ value (the half-maximum effective concentration) of a compound for the degradation of the IRAK3-ePL was calculated using a four-parameter logistic model (sigmoidal dose-response model) (FIT=(A+{(B−A)/1+[(C/x)^{D}]})) where C is the inflection point (EC₅₀), D is the correlation coefficient, and A and B are the low and high limits of the fit, respectively).

The Dₘₐₓ was calculated by determining the maximum percentage loss of target protein following compound treatment.

### Example B2. IRAK Endogenous HTRF Degradation Assay

Cells (˜50 k) were plated in Cisbio 96-well low volume white plates (Cisbio: cat #66PL96005). Compounds were dissolved in DMSO and a 3-fold serial dilution was performed using a TECAN D300E. Cells were incubated with the compound overnight. Total-IRAK3 HTRF kit from Cisbio was used for degradation analysis (Cisbio: 63ADK101PEH). Cryptate and D2 antibodies were diluted in Detection buffer as per manufacturers recommendation. Then 2 μL of each solution was added to 16 μL lysate. Buffer control (lysis buffer detection buffer), Cryptate control (lysis buffer+cryptate antibody+detection buffer), and Negative control (lysis buffer+cryptate antibody+D2 antibody) were made as per manufacturers recommendation. Post incubation with antibodies the HTRF signal was measured using a Perkin Elmer Envision reader and the HTRF signal was calculated using formula: (Emission at 665 nm/Emission at 615 nm)*10,000. All HTRF values were normalized to the average value of DMSO. The average value of the DMSO control wells were set to equal 100% of the relative IRAK3 protein levels. Normalized luminescence values were plotted on a graph as a function of compound concentration. Compound concentration was plotted on the x-axis and the corresponding normalized IRAK3 protein levels on the y-axis. The EC₅₀ value (the half-maximum effective concentration) of a compound for the degradation of the IRAK3 was calculated using a four-parameter logistic model (sigmoidal dose−response model)(FIT=(A+{(B−A)/1+[(C/x)D]})) where C is the inflection point (EC₅₀), D is the correlation coefficient, and A and B are the low and high limits of the fit, respectively). The Yₘᵢₙ was calculated by determining the lowest percentage of target protein remaining following compound treatment. Dₘₐₓ was calculated from Yₘᵢₙ (% Dₘₐₓ=100−Yₘᵢₙ).

### Example B3. IRAK3 Biochemical Binding Assay

The LanthaScreen® Eu Kinase Binding assay was performed as described by the vendor (ThermoFisher Scientific Waltham, MA). Briefly, 100× solutions of compound were prepared in DMSO via serial dilution of the 10 mM stock solution in a 384-well reagent plate using 3-fold intervals to achieve final concentrations. 1 μL of the compound dilution series were added to the corresponding wells of a 384-well reagent plate containing 32.3 μL of 1× buffer (50 mM HEPES pH 7.4, 10 nM MgCl₂, 1 mM EGTA, 0.01% Brij-35). 5 μL of the buffer diluted compounds were transferred to the corresponding wells of a 384-well assay plate. 5 μL of 3× tracer was transferred to each well of the assay plate for a final tracer concentration of 10 nM. Finally, 5 μL of the 3× Eu-Anti-GST and IRAK3 mix was transferred to each well for a final concentration of 2 nM and 10 nM respectively. Reactions were allowed to incubate for 1 hour at room temperature. TR-FRET signal of the interaction (λₑₓ340/λₑₘ 665/λₑₘ 615) was read at room temperature with a delay time of 100 μs and an integration time of 200 μs using an Envision plate reader. Background corrected emission signal ratios at each compound concentration were used to calculate percentage of inhibition (% Inhibition). Plots of % Inhibition versus inhibitor concentrations were fit according to a dose-response equation (Eq. 1) to generate IC₅₀ and Hill slope values using Dotmatics software (Dotmatics, Bishops Stortford, Hertfordshire, England).

$$\% Inhibition = ( \frac{100}{1 + {( \frac{{IC}_{50}}{[ I ]} )}^{n}} ) \qquad ( Eq . 1 )$$

Using these assays, IC₅₀, Dₘₐₓ, EC₅₀, and DC₅₀ values of the following compounds were determined. Dₘₐₓ is defined as the maximum percent degradation achieved and DC₅₀ is the concentration at which 50% degradation is achieved. Data is summarized in Table 2.

TABLE 2

| | | IRAK3 ePL | | IRAK3 HTRF | |
|---|---|---|---|---|---|
| | IRAK3 | degradation | | degradation | |
| Compound | binding | Dₘₐₓ | EC₅₀ | Dₘₐₓ | DC₅₀ |
| No. | IC₅₀ (nM) | (%) | (μM) | (%) | (nM) |
| 1 | 278 | 11 | >10 | 7 | >10000 |
| 2 | 30 | 10 | >10 | 13 | >10000 |
| 3 | 84 | 14 | >10 | 3 | >10000 |
| 4 | 128 | 22 | >10 | 4 | >10000 |
| 5 | 71 | 21 | >10 | 18 | >10000 |
| 6 | 83 | 39 | >10 | 13 | >10000 |
| 7 | 33 | 51 | 1.1 | 27 | >10000 |
| 8 | <0.5 | 78 | 0.017 | 73 | 20 |
| 9 | 214 | 18 | >10 | 33 | >10000 |
| 10 | 236 | 17 | >10 | 30 | >10000 |
| 11 | 5343 | 22 | >10 | 21 | >10000 |
| 12 | 2 | 76 | 0.028 | 81 | 10 |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 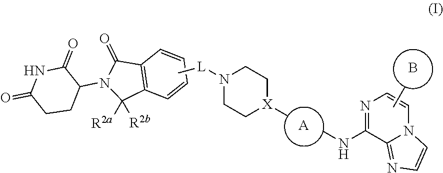
or a pharmaceutically acceptable salt thereof, wherein:
Ring A is C₆-C₁₀ aryl or 5- to 6-membered heteroaryl, wherein the heteroaryl contains 1-3 heteroatoms selected from nitrogen, oxygen, and sulfur;
Ring B is C₆-C₁₀ aryl or C₃-C₆ cycloalkyl, each of which is optionally substituted by 1-5 substituents selected from C₁-C₆ alkyl, C₁-C₆ haloalkyl, C₁-C₆ alkoxy, halo, —OH, and —CN;
X is CH or N;
L is —O(C₁-C₆ alkylene)C(O)—, —O(C₁-C₆ alkylene)C(O)NR¹(C₁-C₆ alkylene)C(O)—, —O(C₁-C₆ alkylene)C(O)NR¹(C₁-C₆ alkylene)-O—(C₁-C₆ alkylene)C(O)—, —O(C₁-C₆ alkylene)C(O)NR¹(C₁-C₆ alkylene)-, or —C(O)(6- to 11-membered spiro heterocyclene)-;
R¹ is H or C₁-C₆ alkyl; and
R^{2a} and R^{2b} are each H or are taken together to form an oxo group.

2. The compound of claim 1, or a pharmaceutically acceptable salt thereof, wherein:
Ring A is C₆-C₁₀ aryl.

3. The compound of claim 1, or a pharmaceutically acceptable salt thereof, wherein:
Ring A is 5- to 6-membered heteroaryl, wherein the heteroaryl contains 1-3 nitrogens.

4. The compound of claim 1 or 2, or a pharmaceutically acceptable salt thereof, wherein:
Ring A is

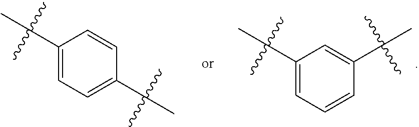 [SMILES: *c1ccc(*)cc1 ; *c1cccc(*)c1]

5. The compound of claim 1 or 3, or a pharmaceutically acceptable salt thereof, wherein:
Ring A is

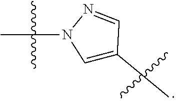 [SMILES: *c1cnn(*)c1]

6. The compound of any one of claims 1-5, or a pharmaceutically acceptable salt thereof, wherein Ring B is C₆-C₁₀ aryl optionally substituted by 1-5 substituents selected from C₁-C₆ alkyl.

7. The compound of claim 6, or a pharmaceutically acceptable salt thereof, wherein Ring B is C₆-C₁₀ aryl optionally substituted by 1-3 substituents selected from C₁-C₃ alkyl.

8. The compound of any one of claims 1-7, or a pharmaceutically acceptable salt thereof, wherein:
Ring B is

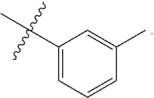 [SMILES: *c1cccc(C)c1]

9. The compound of any one of claims 1-8, or a pharmaceutically acceptable salt thereof, wherein X is CH.

10. The compound of any one of claims 1-8, or a pharmaceutically acceptable salt thereof, wherein X is N.

11. The compound of any one of claims 1-10, or a pharmaceutically acceptable salt thereof, wherein:
L is —O(C₁-C₃ alkylene)C(O)—, —O(C₁-C₃ alkylene)C(O)NR¹(C₁-C₃ alkylene)C(O)—, —O(C₁-C₃ alkylene)C(O)NR¹(C₁-C₃ alkylene)-O—(C₁-C₃ alkylene)C(O)—, —O(C₁-C₃ alkylene)C(O)NR¹(C₁-C₃ alkylene)-, or —C(O)(10- to 11-membered spiro heterocyclene)-; and
R¹ is H.

12. The compound of any one of claims 1-11, or a pharmaceutically acceptable salt thereof, wherein L is:

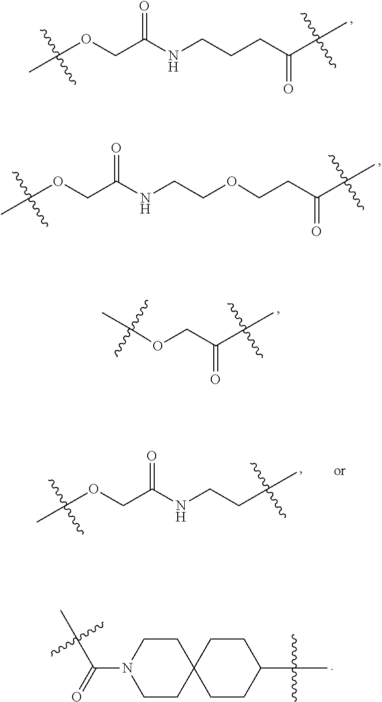 [SMILES: *OCC(=O)NCCCC(*)=O ; *OCC(=O)NCCOCCC(*)=O ; *OCC(*)=O ; *CCNC(=O)CO* ; *C(=O)N1CCC2(CCC(*)CC2)CC1]

13. The compound of any one of claims 1-12, or a pharmaceutically acceptable salt thereof, wherein R^{2a} and R^{2b} are each H.

14. The compound of any one of claims 1-12, or a pharmaceutically acceptable salt thereof, wherein R^{2a} and R^{2b} are taken together to form an oxo group.

15. The compound of any one of claims 1-14, or a pharmaceutically acceptable salt thereof, wherein the compound is Formula (II), (IIIa), (IIIb), or (IV):

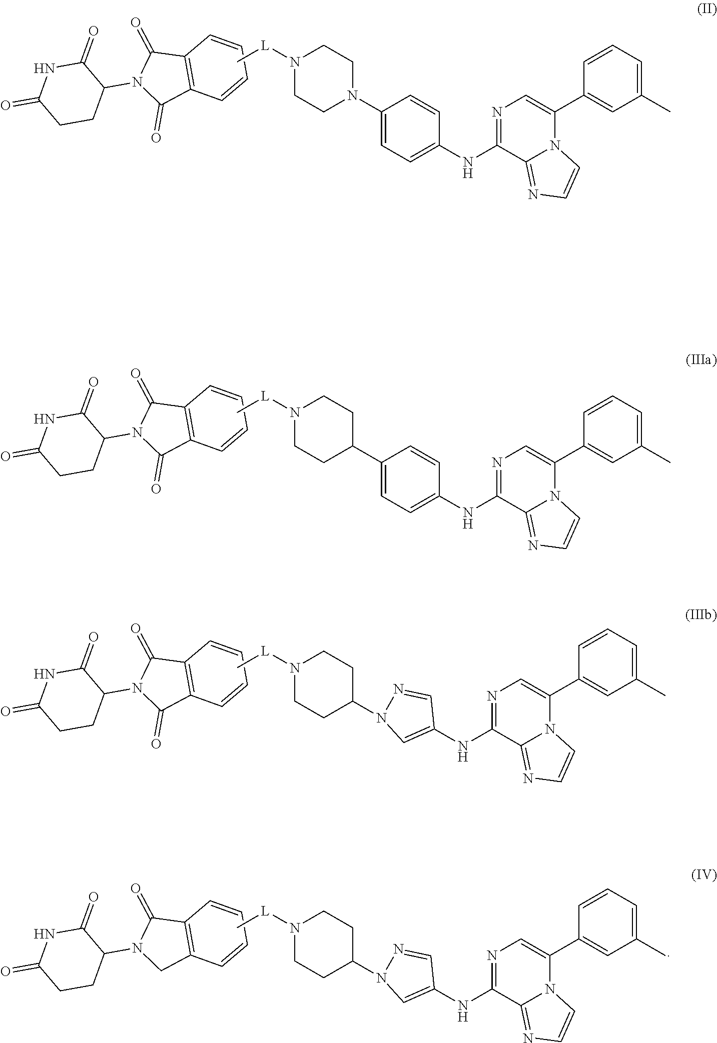

16. A compound selected from the compounds of Table 1 or a pharmaceutically acceptable salt thereof.

17. A pharmaceutical composition comprising the compound of any one of claims 1-16, or a pharmaceutically acceptable salt thereof, and a pharmaceutically acceptable excipient.

18. A method of modulating Interleukin-1 Receptor-Associated Kinase 3 (IRAK3) comprising contacting IRAK3 with an effective amount of the compound of any one of claims 1-16, or a pharmaceutically acceptable salt thereof, or the pharmaceutical composition of claim 17.

19. A method of treating cancer in a subject in need thereof, comprising administering to the subject an effective amount of the compound of any one of claims 1-16, or a pharmaceutically acceptable salt thereof, or the pharmaceutical composition of claim 17, optionally wherein the cancer is selected from bladder cancer, breast cancer, esophageal cancer, colon cancer, head and neck cancer, kidney cancer, lung cancer, pancreatic cancer, prostate cancer, melanoma, and gastric cancer.

20. A method of enhancing immunity in a subject receiving a vaccine, comprising administering to the subject an effective amount of the compound of any one of claims 1-16, or a pharmaceutically acceptable salt thereof, or the pharmaceutical composition of claim 17.
